(19) United States
(12) Patent Application Publication
(10) Pub. No.: US 2024/0198056 A1
(43) Pub. Date: Jun. 20, 2024

(54) APPARATUS AND METHOD FOR SECUREMENT OF A MEDICAL ARTICLE

(71) Applicant: JAVELO HEALTH LIMITED, Edinburgh (GB)

(72) Inventors: Ashton BARNETT-VANES, Edinburgh (GB); Alan KITCHING, Edinburgh (GB)

(21) Appl. No.: 18/288,935

(22) PCT Filed: May 3, 2022
(86) PCT No.: PCT/GB2022/051117

§ 371 Date: Oct. 30, 2023

(30) Foreign Application Priority Data
May 4, 2021 (GB) 2106374.8
Oct. 5, 2021 (GB) 2114275.7

(51) Int. Cl. A61M 25/02

(52) U.S. Cl. CPC A61M 25/02

(57) ABSTRACT

Disclosed is a securement device for securement of a medical article, a kit of parts for a securement device for securement of a medical article; and a method a securing a medical article to a body part. A securement device (100) comprises a medical article engaging member (150) and a body engaging member (140). The medical article engaging member comprises a securing arrangement (130) which defines an open and closed configuration, and a base (120) configured to receive a medical article. In the closed configuration of the securing arrangement (130), the device (100) may prevent rotational, longitudinal and/or lateral movement of a medical article secured within the device (100).

### FIELD OF THE INVENTION

The invention relates to the field of medical article attachment in a clinical setting (e.g. in human or animal healthcare), in particular the securement of invasive devices comprising a conduit (e.g. catheters, vascular access devices, lymphatic access devices, surgical drains, and the like) to a patient in a clinical setting.

### BACKGROUND TO THE INVENTION

Failure of invasive medical articles configured to be inserted in a body lumen such as a vein, artery, lymph node or urinary track is a complex biomechanical and physiological process. Three known failure modes exist and account for invasive medial article (e.g. cannula) failure: blockage of the tubing (40%), its dislodgement out of the body (20%) and leakage (extravasation) of fluid out of the lumen into surrounding tissue (40%). Two major studies published in the leading journal Nature Scientific Reports have undertaken computational and biomechanical analysis of this phenomenon on cannulas (also known as catheters in some countries) that are used to obtain intravascular access in humans or animals. They concluded that mechanical irritation in veins associated with micro-movement of the cannula tip is a key contributor to failure (Piper et al., Scientific Reports. 2018; 8: 3441 and Takahashi et al., Scientific Reports. 2020; 10: 1-13).

While studies have shown that 20% of failure of invasive medical devices carrying a conduit is due to dislodgement/displacement, 80% of cannula failures are due to blockage or leakage of infused material out of the lumen. These occur due to intraluminal irritation from the infused material and the physiological consequences of repeated medical article (e.g. cannula) micromovements as a result of inadequate peripheral securement.

As biomechanical and computational studies show, repeated micromovements of the cannula tubing within the lumen irritates and damages the walls of the vessels and initiates a physiological response that may block the cannula tubing. For example, daily use of invasive medical articles such as cannulas or catheters causes mechanical disruption to the medical article that contributes to failure.

Patients are often mobile and they are unaware or forget about their cannula. They often exert pulling forces which can dislodge the cannula and cause damage to their blood vessels. Moreover, cannulas regularly have fluids such as antibiotics infused in one go (bolus) by hand. When connected to the cannula, the syringe causes a significant moment force or lever which can upend the cannula or contribute to mechanical irritation of the lumen in which the article is inserted (e.g. vein or artery). The connecting of fluid lines also leads to physical disruption of the cannula as luer locks connect via rotation and accordingly lead to a twisting (torque) force being applied to the cannula. Additionally, attachment of luer locks cannulas requires rotational and counter forces on the cannula, which also contribute to irritation of the vein and pistoning.

Cannulas are usually inserted in place and protected with adhesive dressings which provide a good barrier function (e.g. against splashes) to minimise infection of the wound site. However, these adhesive dressings provide poor securement function for the cannulas due to their weak adhesion and further loss of adhesion if the patient has body hair or sweats (this is of particular importance in the veterinary healthcare setting, as animal patients often have fur). In addition, adhesives can cause skin damage, they become tacky when they become wet and cannot be cleaned. In dynamic clinical environments such as pre-hospital response, critical care transfers and emergency response, where timely treatment is vital, the speed at which medical equipment must be connected and used, together with the greater mobility of patients/staff in these settings, and the associated environmental hazards—may place additional forces upon the medical article, exceeding the adhesive properties of standard adhesive dressings.

Existing solutions target specific aspects of intravascular cannula failure. The 2018 The Lancet study (Rickard et al., The Lancet. 2018; 392: 419-430), amongst others, highlighted a major commercial weakness—they have no clear, reproducible effect on increasing cannula survival or dwell times.

Most available stabilisation devices have different models to cater for varying cannula types. Therefore, there is a need for a universal stabilisation device capable of securing medical articles of different sizes (e.g. cannulas of different diameters or forms).

Moreover, the majority of IV cannulas and all radial artery cannulas are inserted parallel to the limb due to the presence of large volume vessels coursing centrally along the upper limb. However, in a significant minority of patients, the cannulated vein may be at an angle. This presents an important technical challenge and therefore there is a need for invasive medical article stabilisation technology which is able to fasten parallel to a body part (e.g. the limb) but contact and secure medical articles (e.g. cannulas) at an angle of between 0 and 90 degrees. While the insertion geometry is usually fixed for radial cannulas (0 degrees), its position in the wrist is not central. For example, existing technology employs adhesive securement solutions applied across the limb to address angled cannulas.

US20140276542A1 discloses a securement device that functions by preventing longitudinal displacement of the cannula which in theory should reduce pistoning within the vein, thereby mitigating the physiological processes that would lead to cannula occlusion. It relies on an adhesive film to adhere the product to the skin—these are notoriously unreliable at maintaining firm patient contact, particularly among those who are unwell and therefore most at risk of and from cannula failure. The device does not secure external appliances connected to a cannula and thus is unlikely to impact on cannula displacement or vein extravasation. Clinical studies have shown this product has no effect on cannula survival (Rickard et al., The Lancet. 2018; 392: 419-430).

Sorbaview SHIELD™ is a dressing-based device that adheres to the skin and, through a reinforced segment, attempts to mitigate the multi-directional movement of cannulas. Adhesive dressings are unreliable and fail at low strains. Patients who are hairy, sweaty or obese are most at risk of cannula failure. The device does not address external appliances connected to a cannula and thus is unlikely to impact on cannula displacement or vein extravasation. A study from 2018 comparing Sorbaview SHIELD to standard care showed no statistically significant difference in cannula survival (Marsh et al., Trials. 2018; 19: 596).

U.S. Pat. No. 9,463,303B2 discloses a securement device for retaining medical tubing, catheters, and other medical fixtures proximal to a patient's skin. The device has a base, at least one channel formed in the base, a flexible strap for overlying the base, and a coupling assembly for coupling the strap to the base. The device is attached to a body part of a patient by means of an adhesive strip, which is likely to fail, particularly on patients with body hair or who are seriously ill and sweat excessively as a consequence.

There is a need for an improved technology that can stabilise invasive medical articles carrying a conduit (such as cannulas), reduce micromovement and eliminate dislodgement for reducing invasive medical article failure and preserving intravascular (e.g. venous or arterial) access.

### SUMMARY OF THE INVENTION

In a first aspect there is provided a medical article securement device. The medical article securement device may comprise a medical article engaging member. The medical article engaging member may comprise a base defining at least one medical article engaging portion (or holder) configured to receive a medical article (or a part or portion of a medical article). The medical article engaging member may comprise a securing arrangement for reversibly securing the medical article to the base. The securing arrangement may define an open configuration and a closed configuration.

The medical article securement device may comprise a body engaging member configured to affix the medical article securement device to a body part of a human or animal.

The medical article securement device may comprise a locking mechanism. The locking mechanism may be configured to allow rotation of the base of the medical article engaging member relative to the body engaging member about a rotational axis of the base when the securing arrangement of the medical article engaging member is in the open configuration. The locking mechanism may be configured to prevent rotation of the base of the medical article engaging member relative to the body engaging member about the rotational axis of the base when the securing arrangement of the medical article engaging member is in the closed configuration.

In use, when a medical article (or portion thereof) is received in the medical article engaging portion and the securing arrangement is in the closed configuration, the securing arrangement may be configured to effect a downward force on the medical article, clamping or otherwise securing the medical article to the base of the medical article engaging member, and an upward force on the base which is configured to prevent the rotation of the medical article engaging member with respect to the body engaging member.

The securing arrangement may be a quick release/quick set securing arrangement. In other words, the securing arrangement may be movable between the open configuration and the closed configuration with a minimum amount of actions. The securing arrangement may be movable between the open configuration and the closed configuration in a maximum of four actions. The securing arrangement may be moved between the open configuration and the closed configuration in a single action. For example, pulling the securing arrangement around the medical article and engaging it with an engagement site on the base may move the securing arrangement from the open configuration to the closed configuration. Releasing the securing arrangement (e.g. in a single action) from the engagement site may move the securing arrangement from the closed configuration to the open configuration. Alternatively, or additionally, pushing on the securing arrangement towards the base until the securing arrangement engages with the base at the engagement site may move the securing arrangement to the closed configuration in a single step or action. Releasing the securing arrangement from the engagement site (e.g. by pushing on a leaver, latch or button or by disengaging the securing arrangement from the base in any suitable way (e.g. undoing a popper, releasing the securing arrangement from a latch or hook, or the like) may move the securing arrangement to the open configuration. The securing arrangement may be moved to the closed configuration by applying a force on the securing arrangement. For example, the securing arrangement may be tightened by applying a pulling force that results in the securing arrangement being stretched, and/or a pushing force on the securing arrangement which causes the securing arrangement to move closer to the medical article (in use). Moving the securing arrangement to the closed configuration (and optionally tightening said securing arrangement around a portion of a medical article) has the following effects: a) decreases the distance between the base and the portion of the medical article engaged with the base (e.g. by depressing the medical article into the base) providing a tighter contact between the medical article and the base and b) pulls the body engaging member towards the base thereby actuating the locking mechanism and preventing rotation of the medical article engaging member with respect to the body engaging member.

Advantageously, the locking action of this invention may synchronise the lock to occur only when an end-user is satisfied with the position of the medical article securement device and the medical article in a single action. This reduces the time taken or delay between setting the medical article securement device and locking it, during which movement and distraction could occur.

The locking mechanism may be reversibly locked and unlocked. Advantageously, this reversible locking action of the locking mechanism ensures that the securing arrangement can be used for different medical articles on the same patient or on a different body part of a patient, or on different patients. This could be beneficial for reducing the carbon footprint and/or cost of the medical article securement device by enabling the device to be re-used.

In some embodiments, only the base of the medical article engaging member may be capable of rotating with respect to the body engaging member. In other embodiments, the complete medical article engaging member (i.e. the base and the securing arrangement), may be capable of rotating with respect to the body engaging member.

In some embodiments, the complete medical article engaging member and/or the base of the medical article engaging member may be capable of rotating 360° with respect to the body engaging portion in the open configuration. Advantageously, this 360° rotation may accommodate infinite medical article orientations as well as the use of the device on left or right limbs.

In other embodiments, the complete medical article engaging member and/or the base of the medical article engaging member may be capable of rotating up to 60°, preferably up to 40°, most preferably up to 30°.

The medical article engaging portion and/or base thereof may be configured to rotate in a clockwise direction only, an anticlockwise direction only, or both.

In preferred embodiments, the medical article engaging portion may be configured to rotate up to 30° in a clockwise direction and up to 30° in an anticlockwise direction. This provides a total of 60° in which the medical article can be adjusted for optimal placing before being secured in the optimal position with the medical article securement device. This arrangement may simplify the device for the end user by limiting the range of rotation so that the device is rotatable within a range that would normally be used in practice, so that it does not complicate assembly with a medical article in use.

The medical article engaging portion and/or base thereof may rotate in predetermined intervals. In other words, the rotation of the medical article engaging portion and/or base thereof may be indexed. For example, the medical article engaging portion and/or base thereof may rotate in 5° to 45° intervals, such as in 5°, or 10°, or 15°, or 20°, or 25°, or 30°, or 35°, or 40°, or 45° intervals. Advantageously, indexing the rotation of the medical article engaging member (and/or base thereof) enables precise and quick fine tuning of the position of the medical article engaging member.

Rotation of the medical article engaging member (and/or the base) with respect to the body engaging member may provide aural feedback, such as audible and/or tactile feedback. For example, rotation of the medical article engaging portion with respect to the body engaging portion may generate a clicking sound and/or a tactile click which may reassure a user that the device is being secured correctly. The aural feedback may be generated every time the medical article engaging portion rotates a predetermined interval or distance as defined above. The aural feedback may be achieved by providing an indexer (e.g. a spring loaded or resilient indexing tab or pole) on the medical article engaging portion (e.g. on the base) such that the indexer moves over a toothed feature (e.g. toothed ring) disposed on the body engaging portion or on an insert to which the body engaging portion is attached. Upon rotation of the medical article engaging portion with respect to the body engaging portion (or insert thereof), the indexer may go over the profile of every tooth and after passing the crest of the tooth move to the gullet of the next tooth providing a clicking sound and feel.

The base may have a top portion configured to interact with a medical article. The top portion may be disposed on an upper surface of the body engaging member. The top portion may comprise or define a first lock component of the locking mechanism and the body engaging member may define a second lock component of the locking mechanism. The first lock component and the second lock component of the locking mechanism may be configured to allow the base to move with respect to the body engaging member when the securing arrangement is in the open configuration. In the closed configuration the first lock component and the second lock component may be configured to prevent movement of the base with respect to the body engaging member (e.g. by engaging the first lock component and the second lock component of the locking mechanism). For example, the base (or the top portion of the base) may define a series of teeth or protrusions and the body engaging member may define a complementary series of teeth or protrusions configured to engage with the teeth or protrusions of the base in the closed configuration of the securing arrangement.

The base may have a top portion and a bottom portion. The top portion may be configured to interact with a medical article. In some embodiments, the bottom portion may be configured to face a body part of a wearer. In other embodiments, the bottom portion may face the body engaging member.

The medical article engaging portion may be configured to receive a medical article (or a part or portion of a medical article). The medical article engaging portion may define a longitudinal axis. In use, the longitudinal axis of the medical article engaging portion may be configured to be aligned with a longitudinal axis of the medical article that is to be received in or coupled to the medical article engaging portion.

The base may define a rotational axis about which the base rotates. The rotational axis of the base may be substantially perpendicular to the body engaging member. The rotational axis of the base may be substantially perpendicular to a longitudinal axis of the body engaging member.

The securing arrangement may define a longitudinal axis. The longitudinal axis of the securing arrangement may be defined along the length of the securing arrangement. In other words, the longitudinal axis of the securing arrangement may be defined between the ends A-A′ of the securing arrangement. The longitudinal axis of the securing arrangement may be substantially perpendicular to the rotational axis of the base when the securing arrangement is in the closed configuration. In some embodiments, the longitudinal axis of the securing arrangement may be substantially parallel to the rotational axis of the base in the open configuration. In other embodiments, the longitudinal axis of the securing arrangement may be substantially perpendicular to the rotational axis of the base in the open configuration. Within the context of this disclosure, substantially parallel may mean disposed at 0°±25° from the rotational axis. Substantially perpendicular may mean disposed at 90°±25° from the rotational axis.

The medical article engaging portion may be disposed along an axis which is substantially perpendicular to a longitudinal axis of the securing arrangement. The medical article engaging portion may be disposed at an angle with respect to the rotational axis of the base. The medical article engaging member may be disposed at an angle with respect to the body engaging member. The medical article engaging portion may define a longitudinal axis which may be disposed at an angle α from about 55° to about 90°, or from about 60° to about 85°, or from about 70° to about 80°, or from about 75° to about 80°, or from about 75° to about 90°, or from about 70° to about 80° from the rotational axis of the base. In some embodiments, the medical article engaging portion may define a longitudinal axis which may be disposed at an angle α from 80° to about 75°, preferably at about 80° from the rotational axis of the base. In other words, the medical article engaging portion may be configured to be inclined with respect to the body engaging member. The medical article engaging portion may define a longitudinal axis disposed from about 5° to about 25°, or from about 10° to about 25°, or from about 15° to about 25°, or from about 20° to about, or from about 10° to about 15°, or from about 5° to about 10° with respect to the body engaging member and/or with respect to a bottom portion of the base of the medical article engaging member. Advantageously, disposing the medical article engaging portion at an angle with respect to the body engaging member (and to the body part of a patient to which the body engaging member is attached in use) may counteract a moment experienced by the medical article (particularly an elongated medical article such as a cannula) where its centre of gravity is positioned at or towards an end portion of the medical article (e.g. the tail end of a cannula) and that centre of gravity together with the residual pull of connected fluid lines predisposes the medical article to tipping backwards, thereby creating an opportunity for back-and-forth micromovements. The angle may be amended depending on the type and dimensions of the medical article that is to be received in the device.

At least a portion of the medical article may be configured to be received in the medical article engaging portion with a longitudinal axis of the medical article being disposed at an angle of from about 0° to about 35°, or from about 0° to about 20°, or about 0° to about 15°, preferably from about 5° to about 10°, further preferably from about 6° to about 10°, or at an angle of about 10° or about 7° with respect to the bottom portion of the base and/or with respect to the body engaging member. The longitudinal axis of the medical article engaging portion may be disposed at an angle of from about 0° to about 35°, or from about 0° to about 20°, or about 0° to about 15°, preferably from about 5° to about 10°, further preferably from about 6° to about 10°, or at an angle of about 10° or about 7° with respect to the bottom portion of the base. The top portion and the bottom portion of the base may be parallel. The top portion may be disposed at an angle with respect to the bottom portion of the base. The angle may be from about 0° to about 35°, or from about 0° to about 20°, or about 0° to about 15°, preferably from about 5° to about 10°, further preferably from about 6° to about 10°, or at an angle of about 10° or about 7°.

The angle between the medical article engaging portion and the bottom portion of the base and/or the body engaging member may be variable. For example, the angle between the medical article engaging portion and the bottom portion of the base and/or the body engaging member may be variable from about 0° to about 35°. The angle between the top portion and the bottom portion of the base may be variable about an axis. For example, the top portion may be reversibly inclined with respect to the bottom portion of the base. The angle between the medical article engaging portion and the bottom portion of the base and/or the body engaging member may be configured to be altered by an end user by any suitable means, such as a hydraulic, pneumatic and/or mechanical arrangement, for example a screw, a pneumatic conduit with a pump or a syringe, or the like. In some embodiments, the top portion of the base or a part thereof may be hingedly coupled to the bottom portion, or to another part of the top portion at one side, while the opposite side may be an unhinged and supported by a mechanical, a hydraulic or a pneumatic arrangement capable of reversibly altering the height of the unhinged part of the top portion. For example, the mechanical arrangement may comprise a screw, the hydraulic or pneumatic mechanism may comprise a fluid receptacle capable of inflating and deflating with a fluid such as air, water, saline solution or the like. Adjustment of the screw or the amount of fluid in the fluid receptacle may alter the height of the unhinged part of the top portion of the base, thus causing the hinged part of the base to rotate about the hinge and altering the angle of the top portion of the base. Advantageously, enabling variability of an angle of the medical engaging portion may maximise comfort of a patient and provide flexibility of use of the device with different types of medical articles. An end user (e.g. a nurse, nursing assistant, doctor or the like) may adjust the angle of the medical engaging portion to ensure that a medical article is received at the optimal angle for a given system.

The top portion of the base may define an upper surface and a lower surface. The medical article engaging portion may be disposed on the upper surface of the top portion of the base. The medical article engaging portion may be defined on the upper surface of the top portion of the base. The lower surface of the top portion of the base may be configured to be parallel to the body engaging member. The lower surface of the top portion may be adjacent to an upper surface of the body engaging member.

The upper surface of the top portion of the base may be parallel to the lower surface of the of the top portion of the base. The upper surface of the top portion of the base may be disposed at an angle from the lower surface of the top portion. The angle may be from about 0° to about 35°, or from about 0° to about 20°, or from about 0° to about 15°, preferably from about 5° to about 10°, further preferably from about 6° to about 8°, or at an angle of about 7°. At least a portion of the top portion may be tapered. For example, the top portion may define a wedge shape.

The medical article engaging member may be configured to prevent longitudinal, rotational and/or lateral movement of a medical article engaged within the medical article engaging portion. Advantageously, the medical engaging member may act as a lock, or a pincer, clamping or otherwise securing a medical article between the base and the securing arrangement and thus preventing displacement of the medical article or rotation of the medical article about a longitudinal axis of the medical article. This is particularly advantageous as the securing arrangement and the base (or medical article engaging portion thereof) together are configured to counteract any force applied on the medical article (e.g. a tugging force, or a rotational force that is sometimes required to be applied in a human or animal healthcare setting).

The medical article engaging portion may be an upstanding wall or projection connected to or extending from the upper surface of the top portion of the base. The upstanding wall may define a shaped surface configured to host a medical article. In use, a medical article is configured to lean against the shaped surface and be secured thereto by the securing arrangement. For example, the upstanding wall may define a curved surface, such as a C-shaped surface, a U-shaped surface, or a V-shaped surface.

The medical article engaging portion may be at least one collar configured to snap fit around a medical article.

The medical article engaging portion may be a channel defined on the top portion of the base. A longitudinal axis of the channel may be disposed at an angle with respect to a plane containing the bottom portion of the base. The angle of from about 5° to about 35°, further optionally wherein the angle is about 15°, or about 10°, or about 7°. The channel may define any suitable shape or form. For example, the channel may define a V-shape, or a U-shape, or a C-shape.

Advantageously, providing a channel, and preferably a V-shaped channel as the medical article engaging portion, may enable universal use of the medical article securement device with medical articles of different sizes as the channel may be capable of wedging or securely hosting the medical articles irrespective of their size. For example, when the medical article is a cannula, the medical article securement device may be able to secure cannulas of different diameters, and therefore be universal. The V-shape may provide at least two defined points of contact between a cannula/NFC of many dimensions and the channel, maximising opportunity for frictional restraint within the channel.

The medical article engaging portion may be a groove. The groove may have any suitable shape. For example, the groove may be C-shaped, U-shaped, or V-shaped.

The securing arrangement may be configured to engage the base and/or the body engaging member by any suitable means. For example, the securing arrangement may be fixedly coupled to the base and/or the body engaging member on one side, but movably coupled on the opposite side, or it may be movably coupled on both sides.

In preferred embodiments, the securing arrangement may be disposed at a normal angle with respect to the medical article engaging portion at all times. Advantageously, this arrangement may provide the maximum clamping force on a medical article received in the medical article engaging portion, thus minimising or eliminating rotational, longitudinal and lateral movements of the medical article even if it is subjected to external forces.

The base and/or the body engaging member may define at least one slit, aperture, or channel configured to receive the securing arrangement and allow movement of the securing arrangement along a longitudinal axis of the securing arrangement through the slit when the securing arrangement is in the open configuration. The securing arrangement may be configured to fit or slide through the slit or aperture.

The slit or aperture may be a hole, a straight slit, or a curved slit, or an L-shaped slit. The base may at least define two apertures connected to each other so as to form a securing arrangement channel. The securing arrangement may be configured to fit or slide through the slit or aperture. The securing arrangement channel may be substantially straight or may define a tortuous path. In some embodiments, the two slits or apertures may be disposed substantially perpendicular with respect to the second of the two slits, thus forming an L-shaped securing arrangement channel. Providing a securing arrangement channel with a tortuous path may be advantageous as it will impart tension on the securing arrangement to avoid or restrict movement of a medical article hosted in the medical article engaging portion when the medical article is subject to a force, such as torque, tension or pulling motion.

In some embodiments, the securing arrangement may slide through the at least one slit, aperture or securing arrangement channel. In order to tighten the securing arrangement, the distance between the medical article engaging portion and the part or section of the securing arrangement disposed opposite (e.g. directly above or across) the medical article engaging portion may be decreased, for example by applying a force on the securing arrangement (e.g. pulling from the securing arrangement along its longitudinal axis, or pushing the securing arrangement towards the base).

The securing arrangement may be coupled (e.g. fixedly or pivotally secured) to the base at one side of the medical article engaging portion. The coupling may be direct or indirect. For example, one end of the securing arrangement may be clamped or otherwise anchored to the base such that it may not move. In some embodiments, the securing arrangement may be pivotally coupled (directly or indirectly) to the base at one side of the medical article engaging portion. For example, one end of the securing arrangement may be pivotally coupled to the base. In some embodiments, one end of the securing arrangement may comprise a feature (e.g. a pin) configured to engage a complementary feature on the base (e.g. an aperture for receiving the pin), so that the base retains the securing arrangement while allowing the securing arrangement to rotate about a pivot axis. In some embodiments, the securing arrangement may be coupled (e.g. fixedly or pivotally coupled) to a yoke which is operatively connected to the base. The yoke may be disposed between the base and the body engaging member. The other end of the securing arrangement may be configured to reversibly engage with the medical article engaging member. For example, the other end of the securing arrangement may be configured to be releasably securable to the medical article engaging member (or base thereof) by any suitable means (e.g. slide through the medical article engaging member and be secured with a ratchet, or go over the medical article engaging portion and engage with the medical article engaging member at the opposite side of the medical article engaging portion to where the securing arrangement is coupled to the base.

In some embodiments, the securing arrangement may be a resilient strap having a first end and a second end. The first end may be fixedly or pivotally secured to the base or a lock component of the locking mechanism. The second end may be configured to engage with an engagement site on the base for reversibly securing the securing arrangement to the base in the closed configuration and not to engage with the engagement site of the base in the open configuration.

The base may define an engagement site for the securing arrangement at the opposite side of the medical article engaging portion (i.e. the opposite side from where the securing arrangement is fixedly or pivotally coupled to the base). The engagement site may comprise any suitable means or arrangement to prevent the securing arrangement from dislodging, releasing or coming off the base. In other words, the engagement site may be any arrangement or means for reversibly securing the securing arrangement to the base. The engagement site for the securing arrangement may be at least one slit, aperture or securing arrangement channel defined on the base at the opposite side of the medical article engaging portion from where the securing arrangement is fixedly or pivotally coupled. The engagement site may be a latch for reversibly securing the securing arrangement to the base. The engagement site may be a resilient tab (e.g. a cantilevered tab) for reversibly securing the securing arrangement to the base. The engagement site may be a leaver for reversibly securing the securing arrangement to the base. The engagement site may comprise an aperture for receiving a part of the securing arrangement and a latch operated by any suitable means (e.g. a button) for reversibly securing the securing arrangement to the base.

In use, a medical article may rest against or be inserted in the medical article engaging portion and the securing arrangement may be disposed over and/or around the medical article (e.g. to prevent dislodgement of the medical article from the medical article engaging portion). The securing arrangement may be configured to releasably engage with the engagement site of the base to reversibly secure the securing arrangement to the base and prevent rotation of the base.

In some embodiments the securing arrangement may be configured to be fed through the at least one slit, aperture or securing arrangement channel disposed at the opposite side of the medical article engaging portion. Once the medical article is located in the correct place and the securing arrangement is tightened/secured, the securing arrangement may be configured to be fastened to minimise or prevent movement of the medical article with respect to the medical article engaging portion.

In some embodiments, the base may define at least one slit, aperture or securing arrangement channel at either side of the medical article engaging portion. In those embodiments, in use, a medical article may rest against or be inserted in the medical article engaging portion and the securing arrangement may be disposed over or around the medical article (e.g. to prevent dislodgement of the medical article from the medical article engaging portion). The securing arrangement may be configured to be fed through each of the at least one slit, aperture or securing arrangement channel disposed at the either side of the medical article engaging portion. In some embodiments, the securing arrangement may be tightened (in use, around the medical article) by pulling the securing arrangement longitudinally outwards from the securing arrangement (in a direction away from the medical article receiving portion) at one or more of the slits. However, in other embodiments, the securing arrangement may be tightened by any other suitable means. Once the medical article is located in the correct place and the securing arrangement is tightened, the securing arrangement may be configured to be fastened to minimise or prevent movement of the medical article with respect to the medical article engaging portion.

The body engaging member may be disposed between the top portion and the bottom portion of the base. Moving the securing arrangement to the closed configuration and tightening said securing arrangement around a portion of a medical article may decrease the distance between the top portion and/or the bottom portion of the base and the body engaging member (providing a tighter contact between the top portion of the base and the body engaging member). Additionally, moving the securing arrangement to the closed configuration (and optionally tightening said securing arrangement around a portion of a medical article) may decrease the distance between the base and the portion of the medical article engaged with the base and may pull the body engaging member towards the base thereby actuating the locking mechanism and preventing rotation of the medical article engaging member with respect to the body engaging member.

The body engaging member may define a lower surface configured to face a body part of a wearer and an opposite upper surface configured to face away from the body part of the wearer. In some embodiments, at least part of the bottom portion of the base may be disposed adjacent to the lower surface of the body engaging member. The top portion of the base may be disposed adjacent to the upper surface of the body engaging member.

The top portion and the bottom portion of the base may be integral. The bottom portion of the base may be configured to snap fit through an aperture defined in the body engaging member. The base may define a recess or groove between the top portion and the bottom portion. The recess or groove may be configured to host a portion of the body engaging portion. In use, the bottom portion of the base may be disposed below the aperture of the body engaging member (i.e. on a lower surface of the body engaging member), the top portion of the base may be disposed above the aperture of the body engaging member (i.e. on an upper surface of the body engaging member), and the recess or groove of the base may be inserted in the aperture of the body engaging member.

The top portion and the bottom portion of the base may be separate parts. The bottom portion may comprise a lower plate configured to be disposed adjacent to the lower surface of the body engaging member and at least one projection (e.g. an arm, protrusion or an upstanding protrusion) configured to engage the top portion of the base above the upper surface of the body engaging member. The top portion of the base may define at least one aperture configured to receive the at least one projection of the bottom portion of the base. In some embodiments, the lower plate of the bottom portion may comprise first and second projections configured to engage the top portion of the base above the upper surface of the body engaging member and the top portion of the base may define at first and second apertures, wherein the first aperture of the top portion is configured to receive the first projection of the bottom portion of the base, and wherein the second aperture of the top portion is configured to receive the second projection of the bottom portion of the base.

The at least one projection of the base may be configured to pass through an aperture of the body engaging member. The at least one projection may be configured to fold over the body engaging member and engage the top portion of the base above the upper surface of the body engaging member.

In some embodiments, the base may comprise only one piece. The base may be disposed adjacent to the upper surface of the body engaging member. The base may be configured to be rotatably coupled to the body engaging member. The base may be configured to snap fit on the body engaging member. The body engaging member may define a lip to receive the base such that the base can be snap fitted to the body engaging member. The snap fit coupling may retain the base on the body engaging member, while enabling rotation of the base with respect to the body engaging member. The base and the body engaging member may be configured to be engaged by means of a circular snap fit arrangement which enables the body engaging member to retain the base while at the same time allowing the base to rotate with respect to the body engaging member.

The device may comprise an additional element between the base and the body engaging member. For example, the device may comprise a lock component between the base and the body engaging member. The lock component may be a yoke. The lock component may be able to pivot, for example from about 3° to about 10°, preferably about 5°. The securing arrangement may be indirectly coupled to the base via the lock component. The lock component may be a yoke defining a feature configured to receive the securing arrangement. For example, the lock component may define a female feature (e.g. an aperture) configured to receive a corresponding male feature of the securing arrangement (e.g. a pin). In some embodiments, the lock component may define a male feature (e.g. a pin or protrusion) configured to be received in a corresponding female feature of the securing arrangement (e.g. an aperture or a feature with a matching shape). The securing arrangement may be pivotally coupled to the lock component (e.g. by engaging the female features and the male features of the securing arrangement and the lock component). The lock component may be operatively coupled to the base. For example, the base may define an aperture or slit through which a portion of the lock component may protrude. The securing arrangement may be coupled to the lock component once the lock component is operatively coupled to the base. The base may comprise a hard material, such as moulded plastic, and/or a resilient material, such as an elastomeric material, synthetic rubber, natural rubber, medical grade silicone.

The top portion of the base may comprise a hard material. The hard material may be lined (at least in the medical article engaging portion) with a soft or resilient material at the medical article engaging portion. For example, the top portion of the base may comprise hard plastics material lined with elastomeric material or silicone. The lining material may be configured to provide friction against the medical article in order to prevent micromovements, as well as to accommodate medical articles of different shapes and/or sizes. Alternatively, the top portion may be moulded in a resilient material, such as rubber, silicone or the like. Advantageously, providing a medical article engaging portion made of or lined with a resilient material or a soft material such as silicone or rubber may maximise the friction coefficient of the material configured to be in contact with a medical article, thus maximising the friction force exerted by the medical article engaging portion on the medical article, and further assisting in the clamping and securement of the medical article within the device to prevent longitudinal, lateral and/or rotational movement of the medical article within the device.

The securing arrangement may take any suitable shape or form. For example, the securing arrangement may be a rigid clamp. The securing arrangement may comprise at least one band. The at least one band may be a flexible strap. The securing arrangement may comprise a plurality of bands (e.g. straps). At least one of the plurality of bands may be fixedly or pivotally attached on one side of the medical article engaging portion and at least one of the plurality of bands may be fixedly or pivotally attached on the other side of the medical article engaging portion. The securing arrangement may be a non-screw fastening securing arrangement.

At least one of the securing arrangement and/or the base comprises a fastener configured to releasably secure the securing arrangement to the base. The fastener may be selected from one or more of: hook and loop fastener, snap-fit, eyelet and pin, hook-and-eyelet, a button and eyelet, a popper, hole and link, slider, clasp, squeeze buckle, D-ring, magnetic snap, cam buckle, ratchet buckle, slide buckle, side release buckle, tie buckle, ratchet teeth and pawl, a latch, a lever, a button for setting/releasing a latch, and the like. In embodiments in which the securing arrangement is fixedly or pivotally attached on one side of the medical article engaging portion, the fastener may be disposed on the opposite side of the medical article engaging portion.

In some embodiments, the securing arrangement may be secured to the base at different lengths. In other words, the securing arrangement may be capable of being tightened and loosened (e.g. by the fastener) at different lengths. The securing arrangement may be capable of being tightened at different predetermined lengths, for example dictated by the distance between ratchet teeth or holes in the securing arrangement. The predetermined length may be from 0.5 mm to about 15 mm, preferably from about 10 mm to about 14 mm. Advantageously, providing a securing arrangement that may be fastened at different lengths enables the medical article securement device to be used with medical articles of different sizes. For example, when the medical article is a cannula, the medical article securement device may be able to secure cannulas of different diameters, and therefore be universal.

In the open configuration, the securing arrangement may be configured to engage the base so as to allow movement of the securing arrangement relative to the base along a longitudinal axis of the securing arrangement. Movement of the securing arrangement relative to the base along the longitudinal axis of the securing arrangement may be configured to modify the distance between the medical article engaging portion of the base and a section of the securing arrangement disposed opposite the medical article engaging portion of the base.

In some embodiments, the securing arrangement comprises a series of ridges that prevent reverse movement in the opposite longitudinal direction of the securing arrangement. For example, in some embodiments, the securing arrangement comprises at least one series of ratchet teeth, ridges or protrusions and the base comprises at least one locking tooth, pawl or indexer configured to releasably secure the securing arrangement and the base and to allow movement of the securing arrangement relative to the base in at least the first longitudinal direction of the securing arrangement. The securing arrangement may be configured to releasably secure a medical article to the base of the medical article engaging member. The securing arrangement may be configured to reversibly lock the rotation (i.e. prevent rotation in any direction) of the medical article engaging member with respect to the body engaging member. When the securing arrangement is subjected to a pulling force along the longitudinal axis of the securing arrangement, the locking tooth, pawl or indexer may allow movement of the securing arrangement with respect to the base in one longitudinal direction of the securing arrangement (i.e. along a first longitudinal direction of the securing arrangement), but not in the opposite direction (i.e. movement in the second longitudinal direction may be prevented). The first longitudinal direction may be along the longitudinal axis of the securing arrangement and away from the base and the second longitudinal direction may be along the longitudinal axis of the securing arrangement and towards the base. Subjecting the securing arrangement to a force (e.g. pulling force) in the first longitudinal direction (i.e. away from the base), may tighten the securing arrangement. When the securing arrangement is subjected to a force in a direction substantially perpendicular to the longitudinal axis of the securing arrangement and towards the base (e.g. when the securing arrangement is depressed towards the base), the securing arrangement may be configured to release the at least one locking tooth, pawl or indexer of the base from the corresponding ratchet tooth, ridge or protrusion and may allow movement of the securing arrangement in the opposite direction (i.e. the second longitudinal direction of the securing arrangement), in order to move the securing arrangement to the open configuration. In some embodiments, a channel or slit of the base acts as the pawl, locking tooth or indexer of the securing arrangement.

The fastener of the securing arrangement mechanism may be reversibly locked and/or tightened and unlocked/released/loosened. Advantageously, this reversible fastening action of the securing arrangement ensures that the securing arrangement can be used for different medical articles (e.g. medical articles of different sizes) on the same patient or on a different body part of a patient, or on different patients. This could be beneficial for reducing the carbon footprint and/or cost of the medical article securement device by enabling the device to be re-used. It may also provide a high degree of control to the end user (e.g. nurse) to ensure that the fastener can be adjusted based on end user experience or patient feedback (e.g. tighter or looser).

In some embodiments, the securing arrangement may comprise at least one hole or a plurality of holes spaced apart along a longitudinal axis of the securing arrangement. The base may comprise at least one hook, protrusion or wing. In the closed configuration, the hook may be configured to engage the at least one hole of the securing arrangement to prevent movement of the securing arrangement relative to the base along the longitudinal axis of the securing arrangement. In the open configuration, the hook may be configured not to engage any hole of the securing arrangement.

In the closed configuration, the securing arrangement may be configured to subject the base of the medical article engaging member to a pulling force away from the body engaging portion. In other words, in the second configuration, the securing arrangement may pull the base upwards (in a direction away from the body engaging member). In the closed configuration, the securing arrangement may be configured to subject a medical article disposed on the base to a pushing force towards the base.

The securing arrangement may comprise a flexible material. The securing arrangement may comprise a wipe-clean material. The securing arrangement may comprise a biodegradable material and/or a recyclable material. The securing arrangement may comprise one or more materials selected from: flexible plastic, an elastomeric material, synthetic rubber, natural rubber, medical grade silicone, natural fabric, synthetic fabric, and similar materials thereof. The securing arrangement may comprise a hard material. For example, the securing arrangement may comprise a hard plastic. In use, the securing arrangement may comprise a flexible but inelastic material. Hard or inelastic materials may be able to strongly withstand loading forces on the medical article. However, they cannot conform to different sizes or shapes of medical articles. For example, in embodiments in which the securing arrangement is a clamp, the clamp will only be able to accommodate medical articles of a specific size/shape or of sizes with minor variability in size/shape. A clamp arrangement can be very quickly set and unset and can provide a very strong securement of a medical device to prevent movement or dislodgement from the device.

The securing arrangement may comprise a resilient material. The resilient material may be configured to mould and/or grip around a medical article (in use). Advantageously, a resilient material may be capable of adapting to the contour of medical articles of different shapes and/or sizes while simultaneously have the resilience to not elastically deform to a loading force on the medical article (e.g. upward and/or backward deflection), such that it retains the medical article secured in the device. Securing arrangements with resilient materials are advantageous as they can also be deployed (set/unset) quickly and they are adaptable to different shapes and sizes of medical articles, making the device more universal (one size fits all).

The securing arrangement may comprise a mixture of materials. For example, the securing arrangement may comprise a hard material and a resilient insert to increase the grip of the hard material and accommodate for small variations in shape and/or size of the medical article. In some embodiments, the securing arrangement may be a clamp comprising a hard material (e.g. plastic) lined with a resilient insert (e.g. silicone) to increase the grip of the medical article in use. The securing arrangement may comprise an overmould element (e.g. a pin or a protrusion) made of a hard material for securely engaging with the base of the medical article engaging portion and/or the locking mechanism. The securing arrangement may also comprise a resilient material securely engaged with the overmould element in order to be able to engage with and secure a medical article. This arrangement confers the securing arrangement with the adaptability of a resilient material (e.g. in a strap) and the required strength for a strong coupling to the medical article engaging member and/or locking mechanism. In some embodiments, the securing arrangement may comprise a small overmould secured to an otherwise unreinforced resilient material to form a strap. In other embodiments, the securing arrangement may comprise a rib of a hard but flexible material running along the underside perimeter of the securing arrangement to provide a grip feature against the contour of the medical article. In yet other embodiments the securing arrangement may comprise a semi-rigid (e.g. hard but flexible) reinforcing member (e.g. a ladder structure) embedded within a resilient material (e.g. strap). The reinforcing member may reduce the stretch properties of the resilient material and increase its capacity to withstand loading forces in use.

In some embodiments, the securing arrangement may comprise a resilient material, such as silicone or rubber. Advantageously, securing arrangements comprising or made of a resilient material may have a dampening or shock absorbing effect on any force exerted on a medical article which is secured in the medical article securement device during use. Additionally, the resilient material may maximise the friction coefficient of the material of the securing arrangement, thus maximising the friction force exerted by the securing arrangement on the medical article, thus further assisting in the clamping and securement of the medical article within the device to prevent longitudinal, lateral and/or rotational movement of the medical article within the device.

In some embodiments, the securing arrangement may be configured to reversibly engage with a latch or resilient tab of the base (in the closed configuration). For example, when moving from the open configuration to the closed configuration, the securing arrangement may be configured to press against the resilient tab, displace the resilient tab and be latched under the resilient tab. The securing arrangement may be released from the latch or resilient tab by deflecting the latch or resilient tab.

In some embodiments, the securing arrangement may be a clamp or a snap cap. The clamp may be sized to fit around and secure a medical article of a specific size. The securing arrangement (clamp or snap cap) may be pivotally or hingedly coupled to the base at one end of the securing arrangement (clamp or snap cap). The other end of the securing arrangement is configured not to engage with the base in the open configuration and to reversibly engage with the base in the closed configuration. The base may define or comprise any suitable means for pivotally or hingedly securing one end of the clamp or snap cap to the base. For assembling the securing arrangement and the base, an end of the securing arrangement may be configured to snap on the base. For example, the base may define a recess, a protrusion or a hook configured to host a portion of the clamp or snap cap and retain it in place while allowing pivotal movement of the clamp or snap cap relative to the base. The base may define a recess, a protrusion or a hook at one side of the medical article engaging portion configured to receive an end of the clamp or snap cap and pivotally engage said end of the clamp or snap cap.

The base may define or comprise any suitable means for reversibly securing the clamp to the base in the closed configuration. For example, the base may define a latch or resilient tab for reversibly securing the clamp to the base in the closed configuration. Moving the clamp from the open configuration to the closed configuration, a free end of the clamp (or snap cap) (i.e. the end which is not hingedly coupled to the base) may be configured to press against the latch or resilient tab, deflect it in order to move under the latch or resilient tab and be retained therein. The latch or resilient tab may be biased towards a position in which the portion of the latch or resilient tab which engages with the clamp is closer to the bottom portion of the base (i.e. lower and/or closer to the locking mechanism) than the position of the portion of the latch or resilient tab which engages with the clamp when the tab or resilient tab is being deflected to allow the clamp to pass clear of the latch or resilient tab (for example to move from the open configuration to the closed configuration and vice versa). Pressing the clamp against the resilient tab (or latch) deflects the resilient tab (or latch) to allow the resilient tab to pass under the resilient tab. When the clamp reaches the closed configuration, the resilient tab springs back to its original position, thus preventing the clamp from inadvertently/accidentally releasing and opening. Therefore, to move from the open configuration to the closed configuration, the clamp may be pivotally moved and pressed against the means for securing the clamp to the base (i.e. resilient tab or latch). The means for securing the clamp to the base may move to allow the clamp pass clear of said means for securing the clamp, and spring back to its original position, thus locking the clamp into place. Therefore, the clamp may be snap fit (or quick-set) into the closed configuration. In addition, release of the clamp can be easily achieved and the engagement site provides a quick release that can release the clamp easily when a user actuates the engagement site, but does not do so accidentally (just by a patient pulling or touching the device).

The clamp or snap cap may comprise a hard or rigid material. For example, the clamp or snap cap may comprise a metal, a rigid polymeric material, such as any suitable plastic, or the like. The material of the clamp or snap cap may be rigid enough to withstand the forces to which it would be subjected by a user to push the clamp or snap cap to move it to the closed configuration and to resist any pulling forces exerted by pulling or tugging a medical article secured within the device. Advantageously, providing a clamp or snap cap comprising a rigid material as a securing arrangement provides a stronger securement against vertical displacement of the medical article when it is secured in the closed configuration of the medical securement device.

The clamp or snap cap may comprise a resilient insert (e.g. made of rubber, silicone, medical grade silicone, resilient polymeric material, or the like). The resilient insert may be configured to be in contact with the medical article when the clamp or snap cap is in the closed configuration. The resilient insert may allow the clamp or snap cap to secure medical articles which have slight variations in size (e.g. 0.1-2.5 mm variations in size). The resilient insert may have a high friction coefficient. The resilient insert may be configured to absorb forces exerted on the medical article by movement of the patient to which the device and medical article are secured, and may resist movement of the medical article within the device.

The clamp or snap cap may comprise a resilient material disposed on a top surface (which in use would be facing away from the medical article). The resilient insert of the clamp or snap cap may be over moulded or extend through the material of the clamp/snap cap.

Advantageously, the resilient material disposed on a top side of the clamp/snap cap may provide a textured touch point (e.g. soft/rubbery) on the upper side of the securing arrangement (clamp/snap cap). This may provide a clear tactile indication to the end user on where to push to close the securing arrangement and may assist patients with visual impairment who rely on tactile features. The resilient material disposed on the top surface of the clamp/snap cap may comprise a luminescent material or a luminescent colouring. This may improve low-light visualisation of the medical article securement device (e.g. at night, or in low light conditions which are often encountered in the field).

In use, securing the clamp (or snap cap) to the base prevents longitudinal, rotational and/or lateral movement of a medical article engaged within the medical article engaging portion and prevents rotation of the base about the rotational axis of the base relative to the body engaging member. In the open configuration of the clamp, the base may be allowed to rotate about its rotational axis. The rotation may be a free rotation or an indexed rotation (i.e. the base may be allowed to rotate in predetermined intervals). In embodiments in which the rotation is indexed, the user may receive aural indication that the base is being rotated (e.g. a clicking sound and/or feel).

Advantageously, the clamp enables a fast and quick securement action which can be performed with one hand. This may minimise micromovements of a medical article while it is being secured to the medical article securement device described herein. The snap fit closure mechanism of the clamp is quick to deploy with a single hand, secures a medical article against unwanted releases of the medical article which may happen when a patient moves the body part to which the device is attached, and it is easy to revert back to the open position by a medical practitioner when the medical article needs to be released.

In some embodiments, in the open configuration of the securing arrangement, the locking mechanism may allow rotation of the medical article engaging member in one direction (e.g. clockwise or anticlockwise), but not in the opposite direction. In other embodiments, in the open configuration of the securing arrangement, the locking mechanism may allow rotation of the medical article engaging member in a first and a second directions (e.g. clockwise and anticlockwise). In the closed configuration of the securing arrangement, the locking mechanism may prevent rotation of the base of the medical article engaging member in either direction.

The locking mechanism may comprise a first lock component and a second lock component. The first and second lock components may be complementary. For example, the first lock component may define a shape complementary to the second lock component. The first and second lock components may be configured to be engaged in the closed configuration. The first and second lock components may be configured not to engage in the open configuration. The distance between the first lock component and the second lock component may be configured to be greater when the securing arrangement is in the open configuration than when the securing arrangement is in the closed configuration. Without wishing to be bound by theory, the smaller distance between the first lock component and the second lock component may activate the lock mechanism and prevent movement of the medical article engaging member with respect to the body engaging member.

In some embodiments, the first lock component may be disposed on the base and the second lock component may be disposed on the body engaging member or on a platform or insert configured to be coupled to or be part of the body engaging member. In some embodiments, the first lock component may be disposed on the securing arrangement, and the second lock component may be disposed on the body engaging member or on a platform or insert configured to be coupled to or part of the body engaging member. In some embodiments, the first lock component may be disposed on an insert disposed between the base and the body engaging member, and the second lock component may be disposed on the body engaging member or on a platform or insert configured to be coupled to or be part of the body engaging member. In some embodiments, the first lock component may be disposed on the base and the second lock component may be disposed on an insert disposed between the base and the body engaging member. Within the context of this disclosure, first or second lock components may be interchangeable.

The locking mechanism may comprise at least one of: a frictional lock between the lock components, a unidirectional or bi-directional ratchet mechanism, a series of teeth in the first lock component and a pawl (or locking tooth, or indexer) on the lock component, a series of teeth in the first lock component and a corresponding series of teeth on the second lock component, at least one male feature (e.g. a post) in the first lock component and a corresponding female feature (e.g. an aperture, a slot or a hole) in the second lock component.

The first lock component may comprise a toothed feature and the second lock component may comprise a corresponding toothed feature configured to engage with the toothed feature of the first lock component. The first lock component may comprise at least one male feature (e.g. a protrusion or a post) and the second lock component may comprise a female feature (e.g. a hole, slot, or aperture) configured to receive and engage with the male feature of the first lock component.

The toothed features of the first and second lock components may be complementary and optionally they may be tapered. Providing tapered teeth may confer the lock with an improved mating of the first and second lock components as engagement occurs moving to the closed configuration (i.e. the tapered teeth of the first and second lock components will positively engage each other. In other words, if the tapered teeth have are about to bounce off with respect to each other the tapered shape makes it more likely that the teeth will ‘slide’ into engagement under the downward pressure exerted by the securing arrangement. This means that for an end-user there is likely to be quick if not instantaneous locking/engagement. In addition, the tapered teeth provide improved clearance for rotation between the first and second lock components in the open configuration. This will provide a stronger lock in which the toothed features of the first and second components of the lock are less likely to engage negatively (i.e. get jammed).

The locking mechanism may comprise a locking element disposed between the base of the medical article engaging member and the body engaging member. The locking member may be operatively coupled to the base of the medical article engaging member and to the securing arrangement. The locking element may comprise a first lock component configured to engage with a second lock component disposed on the base or on the body engaging member when the securing arrangement is in the closed configuration and not to engage with the second lock component when the securing arrangement is in the open configuration. The locking member may be configured to pivot between a first position when the securing arrangement is in the open configuration and a second position when the securing arrangement is in the closed configuration. The locking member may engage with the base and/or the body engaging member in at least two opposing locations.

In embodiments in which the securing arrangement is a clamp or a snap cap, the base may comprise the first lock component disposed at a distance from the second lock component. The first lock component may be a toothed resilient tab or cantilever arm extending substantially parallel to the body engaging member and at a close distance from the second lock component, but allowing free rotation of the base with respect from the body engaging member when the clamp (or snap cap) is in the open configuration. The second lock component may be a toothed feature configured to engage with the toothed feature of the first lock component. The second lock component may be a toothed ring. The toothed ring may be disposed on the body engaging member. The toothed ring may be disposed on an insert disposed between the body engaging member and the base. The toothed ring may be disposed on an insert or base to which the body engaging member is configured to be attached. For example, the insert or base may comprise slots or holes through which the body engaging member (e.g. a strap) may be fed for securing the body engaging member to the insert or base. Pressing on the clamp may subject the clamp to pivotal movement and the end of the clamp which is not pivotally coupled to the base may press against the first lock component and deflect it until the teeth of the first lock component engage with the teeth of the second lock component. The clamp may be secured in the closed configuration by a second resilient tab or cantilever arm which may act as a latch to prevent the clamp from accidentally moving to the open configuration. Therefore, in the closed configuration, a medical article disposed in the medical article engaging portion is pinched between the clamp and the medical article engaging portion and thus prevented from lateral and/or rotational movements. Simultaneously, moving the clamp to the closed configuration, actuates the locking mechanism and prevents rotation of the medical article engaging member with respect to the body engaging member. To move the clamp to the open position, latch (or resilient tab) is deflected to provide clearance for the clamp to pivot back to the open configuration. This in turn causes the resilient tab (or cantilever arm) to spring back to its original position which is not in engagement with the teeth of the second lock component, thus allowing the medical article engaging member to rotate again.

In other embodiments in which the securing arrangement is a clamp or a snap cap, the first lock component may be disposed on a locking insert located between the clamp (or snap cap) and the body engaging member or insert thereof. The locking insert may comprise a cantilevered tab comprising a toothed feature or profile. The second lock component may be a toothed feature configured to engage with the toothed feature of the first lock component. The toothed features of the first lock component and the second lock component may be angled or tapered and complementary to each other. The second lock component may be a toothed ring. The toothed ring may be disposed on the body engaging member. The toothed ring may be disposed on an insert disposed between the body engaging member and the base. The toothed ring may be disposed on an insert or base to which the body engaging member is configured to be attached. For example, the insert or base may comprise slots or holes through which the body engaging member (e.g. a strap) may be fed for securing the body engaging member to the insert or base.

In some embodiments, in the open configuration, the toothed feature of the first lock component may be configured to engage with the toothed feature of the second lock component. Subjecting the medical article engaging member to a rotational force in the open configuration may force a deflection of the cantilevered tabs and may allow rotation with a clicking sound. Pressing on the clamp may subject the clamp to pivotal movement and the ends of the clamp may press against the locking insert(s) and deflect it/them until the teeth of the first lock component intimately engage with the teeth of the second lock component. In some embodiments, there may be two inserts comprising the first lock component disposed at either side of the medical article engaging portion of the base. In other words, the two locking inserts may be diametrically opposed. Advantageously, this arrangement may allow for opposing engagement forces of the first lock component with the second lock component when the clamp is in the closed configuration. Therefore, this arrangement may provide a stronger lock that is less likely to fail under strain.

In other embodiments, the locking insert or inserts is/are disposed within a tapered pocket of the body engaging portion or insert thereof. The locking insert(s) may have a boss matching the shape of the tapered pocket. This configuration may provide a clearing distance between the first lock component and the second lock component in the open configuration. In other words, in the open configuration, the toothed features of the first lock component and the second lock component are not engaged. When moving to the closed configuration of the clamp (or snap cap), the ends of the clamp press against the locking inserts, deflecting the cantilevered portion and sliding the boss along the tapered pocket until the teeth of the first lock component and the second lock component engage. Advantageously, this configuration provides maximum engagement between the toothed interfaces/features of the first and second lock components in the closed configuration. This configuration also increases the height of the teeth, which allows for greater engagement and therefore for a stronger lock.

The clamp may be secured in the closed configuration by a resilient tab or cantilever arm which may act as a latch to prevent the clamp from accidentally moving to the open configuration. Therefore, in the closed configuration, a medical article disposed in the medical article engaging portion is pinched between the clamp and the medical article engaging portion and thus prevented from lateral and/or rotational movements. Simultaneously, moving the clamp to the closed configuration, actuates the locking mechanism and prevents rotation of the medical article engaging member with respect to the body engaging member. To move the clamp to the open position, latch (or resilient tab) is deflected to provide clearance for the clamp to pivot back to the open configuration. This in turn causes the resilient tab (or cantilever arm) to spring back to its original position which is not in engagement with the teeth of the second lock component, thus allowing the medical article engaging member to rotate again.

In further embodiments in which the securing arrangement is a clamp or a snap cap, the clamp may comprise the first lock component. The first lock component may be a toothed feature/interface disposed on one or on both ends of the clamp. For example the clamp may comprise a toothed feature at an end which is configured to engage the lock component at one side of the medical article engaging portion and/or a second toothed feature at the opposite side of the medical article engaging portion. In embodiments in which the clamp comprises toothed features disposed in two locations disposed at either side of the medical article engaging portion may allow for opposing engagement forces with the second lock component when the clamp is in the closed configuration. Therefore, this arrangement may provide a stronger lock that is less likely to fail under strain. The first lock component may comprise a male feature. For example, the first lock component may comprise at least one peg or post protruding from the clamp. The male feature may be disposed on the clamp at one or at both ends and it may be configured to engage a corresponding female feature on the second lock component to prevent rotation of the medical article engaging member relative to the body engaging member. In some embodiments, the first lock component comprises both at least one toothed feature and at least one female feature. This may allow for stronger engagement between the first and second lock components and therefore may create a stronger lock.

The second lock component may be a toothed feature/interface configured to engage with the toothed feature of the first lock component. The second lock component may be a toothed ring. The second lock component may be a female feature (e.g. hole, slot or aperture) configured to receive a male feature of the first lock component. The toothed ring and/or the female feature may be disposed on the body engaging member. The toothed ring and/or the female feature may be disposed on an insert disposed between the body engaging member and the base. The toothed ring and/or the female feature may be disposed on an insert or base to which the body engaging member is configured to be attached. For example, the insert or base may comprise slots or holes through which the body engaging member (e.g. a strap) may be fed for securing the body engaging member to the insert or base.

Pressing on the clamp may subject the clamp to pivotal movement and the first lock component or components of the clamp may engage with the second lock component or components of the body engaging member or insert of the body engaging member. For example, in embodiments in which the first lock component is a toothed feature and the second lock component is a toothed ring, moving the clamp to the closed configuration may cause the teeth of the clamp to engage the toothed ring of the body engaging member (or insert thereof) and prevent rotation of the medical article engaging portion. In embodiments in which the first lock component is a male feature and the second lock component is a female feature (or vice versa), moving the clamp to the closed configuration may engage the male feature with the female feature. In embodiments in which the first lock component comprises a toothed feature and a male or female feature and the second lock component comprises a complementary toothed feature (e.g. toothed ring) and a complementary female or male feature, the lock may be stronger and may withstand pulling or tugging forces. In embodiments in which the clamp comprises the first lock component or components at both ends such that in the closed configuration the lock components engage with the corresponding second lock components at either side of the medical article engaging portion, the opposite engagement forces may provide a stronger lock.

The clamp may be secured in the closed configuration by a resilient tab or cantilever arm which may act as a latch to prevent the clamp from accidentally moving to the open configuration. Therefore, in the closed configuration, a medical article disposed in the medical article engaging portion is pinched between the clamp and the medical article engaging portion and thus prevented from lateral and/or rotational movements. Simultaneously, moving the clamp to the closed configuration, actuates the locking mechanism and prevents rotation of the medical article engaging member with respect to the body engaging member. To move the clamp to the open position, latch (or resilient tab) is deflected to provide clearance for the clamp to pivot back to the open configuration. Pivoting the clamp to the open configuration causes the first lock component to disengage from the second lock component, thus allowing the medical article engaging member to rotate again.

The body engaging member may be capable of securing the medical article securement device to a body part of a human or animal. The body part may be any body part, such as a torso, neck, limb, hand, paw, finger, toe, thorax, abdomen, head, groin, armpit, reproductive organ, or the like. The body part may be a limb. The body part may be an arm or a leg. The body part may be configured to be located anywhere in the upper arm, the hand, the lower arm, the upper leg, the lower leg, or the foot. The body part may be a hand, a wrist, an elbow, a foot, an ankle, a calf, a thigh, a groin. The body part may be an upper arm. The medical article securement device may be capable of being secured to both a left and a right limb. In other words, the medical article securement device may be ambidextrous. For example, the medical article securement device may be capable of being secured to both of the left arm and the right arm, the left hand and the right hand, the left leg and the right leg, or the left foot and the right foot.

The body engaging member may have any suitable shape or form. In some embodiments, the body engaging member may comprise an adhesive. In other embodiments, the body engaging member may not comprise an adhesive. For example, the body engaging member may comprise a strap (e.g. one or more straps), an adhesive strip, a sleeve, a hook, a clip, a clamp, a bracelet, or the like. The body engaging member may be generally elongate. The body engaging member may be longer than it is wide. The body engaging member may define a non-radial shape. Advantageously, providing body engaging member with generally elongate or non-radial shapes may alloy the body engaging member to wrap around a body part and firmly secure the body medical article securement device to the patient.

In preferred embodiments, the body engaging member may not comprise an adhesive. Advantageously, providing a medical article securement device that does not rely on adhesive for affixing the device to a body part provides reliable securement of a medical article in all conditions, in particular in high humidity conditions. This is particularly important when the medical article securement device is used on very sick patients, who often show high rates of perspiration, or in high ambient humidity conditions. Humidity may render adhesive tacky and decrease the adhesive's effectiveness. In addition, avoiding the use of adhesive may minimise allergic reactions or skin hypersensitivity in patients with sensitive skin.

The medical article securement device may comprise any suitable material. The medical article securement device may comprise a wipe-clean material. The medical article securement device may tolerate antiseptic solvents, such as alcohol-based solvents. Advantageously, this may allow alcohol-wipe based cleaning to disinfect the device prior to or during use. Advantageously, employing wipe clean materials ensures that the medical article securement device can be worn for a long period of time without the risk of infections or dirt build-up to the wearer. Even if the medical article securement device becomes dirty (e.g. with biological fluids, medicaments, sweat, or spillages), the device may be wipe-cleaned and used by the wearer until the device is no longer required.

The medical article securement device may comprise a medical grade material. Employing medical grade materials may minimise the risk of infection and allergies suffered by a wearer as a result of wearing the medical article securement device.

The medical article securement device, or one or more component parts thereof may be made from a recyclable material, such as a thermoplastics material, or a biodegradable material, such as a biodegradable plastics material. Biodegradable plastics materials, as a cellulosic or other plastics materials based upon plant-based polymers or resins are known in the art. Advantageously, manufacturing the medical article securement device with recyclable or biodegradable materials reduces the environmental impact of the devices, and therefore enables the medical article securement device to be used as a disposable item, which is of great convenience in the human or animal healthcare setting where cross-infection between patients needs to be minimised.

The body engaging member may comprise a fastener to secure the medical article securement device around the body part. The fastener may have any suitable shape or form. For example, the fastener may be selected from one or more of: hook and loop (e.g. Velcro®), snap-fit, eyelet and pin, hook-and-eye, hole and link, slider, clasp, squeeze buckle, D-ring, magnetic snap, cam buckle, ratchet buckle, slide buckle, side release buckle, tie buckle, or any other mating/interfacing feature known in the art.

The body engaging member comprise a flexible material. The body engaging member comprise a resilient material. The body engaging portion may comprise a wipe-clean material. The body engaging member may comprise a biodegradable material and/or a recyclable material. The body engaging portion may comprise a soft material. The body engaging portion may comprise a resilient material. The body engaging portion may comprise a stretchable material.

The body engaging portion may comprise one or more materials selected from: flexible plastic, an elastomeric material, synthetic rubber, natural rubber, medical grade silicone, natural fabric, synthetic fabric, a porous material. In preferred embodiments, the body engaging portion may comprise silicone.

Without wishing to be bound by theory, providing a body engaging member having a soft, stretchable and/or resilient material facing a wearer's skin may be maximise the comfort of the wearer, and maximise the grip of the body engaging member on a patient's body part.

The body engaging member may comprise one or more ventilation apertures to minimise accumulation of perspiration when a patient wears the device. In some embodiments, the fastener of the body engaging member is a buckle configured to fit through and secure one or more apertures of the body engaging member. The apertures of the body engaging member double up as part of the fastening means of the body engaging member as well as ventilation apertures.

The body engaging member may comprise a reinforced segment resistant to stretching. The reinforced segment may have lower stretching than the unreinforced portions of the body engaging member. The reinforced segment may comprise a rigid material, such as moulded plastic (e.g. polypropylene). The reinforced segment may be sufficiently flexible to be curved around a portion of a patient's limb or other body part when the device is attached thereto, but which is substantially undeformed under the action of the lateral forces applied between the straps, in use.

Advantageously, the reinforced segment may prevent a 360° tourniquet effect (i.e. preventing blood flow) on the body part of the wearer to which the medical article securement device is attached.

The second lock component may be defined in the reinforced segment.

The body engaging member may comprise a lining configured to face the skin of a wearer. The lining may comprise a soft material. The lining may comprise a resilient material. Without wishing to be bound by theory, providing a body engaging member having a lining comprising soft or resilient material facing a wearer's skin may enable the use of any suitable material for the body engaging member, while maximising the wearer's comfort and preventing tourniquet effect on the body part of the wearer to which the medical article securement device is attached.

The size of the body engaging member may be variable. The body engaging member may be adjustable to fit different sizes of body parts. For example, the body engaging member may be an adjustable strap configured to fit most sizes. The body engaging member may be available in universal size or in adjustable adult and child size. The size of the body engaging member may be variable to fit around the circumference of an upper limb circumference of slim to obese patients.

The medical article may be an invasive device comprising a conduit. The medical article may be selected from: a catheter, a cannula, a vascular access device, a needle free connector (NFC) for a cannula, a needle free connector-fluid line connector (e.g. luer connector) junction, a lymphatic access device, a peripheral vascular access device, a needle, such as a blood transfusion and/or a dialysis needle, a central vascular access device, an arterial vascular access device, a venous vascular access device, a line, a midline, a peripherally inserted central catheter (PICC), a central arterial access device, a central venous access device, a central venous catheter (CVC), a subclavian line, an internal jugular line, a femoral line, a urinary catheter, a surgical drain.

The medical article securement device or component parts thereof may be provided with a luminescent coating or may be impregnated with a luminescent pigment or dye. This may assist for example in locating the medical article securement device at night or in low light conditions, or may serve as a reminder to a patient that they are still wearing the medical article securement device.

Advantageously, the medical article securement device is a non-invasive medical device that is capable of effectively securing medical articles and promoting their longevity by collectively addressing the biomechanical factors that impact on invasive medical article disruption or failure.

The medical article securement device may secure the medical article (e.g. a cannula) in its correct position on a body part (e.g. upper limb), provide a fixed platform for medical article use, and improve patient awareness of the medical article through enhanced proprioception.

The medical article securement device may enable visualisation of and access to the relevant portions of the medical article when it is secured to a body portion. For example, in embodiments in which the medical article is a cannula, a luer lock portion of the cannula or a luer or needle free connector may be secured to the medical article securement device while allowing access and visualisation of a luer or needle free connector, such as a BD Smart Site™, for example to adjust or change tubing, the fluids that need to be inserted via the cannula, cleaning etc.

The medical article securement device, once assembled, may be a single unit. In other words, once assembled for use, the medical article securement device may not comprise different parts or portions which need to be assembled together or which can easily dislodge or come off from the device. This is desirable in the high-pace medical settings where an end-user needs to quickly secure a medical article to a body part and does not have time to fiddle with multiple parts of a securement device. Additionally, and unlike devices with multiple parts, the unitary (or single unit) medical article securement device may be less prone to contamination from a part of the device dislodging and falling to the floor.

The device may be capable of integrating with dressings, such as adhesive dressings (e.g. Tegaderm™).

The device may be universal. The device may be configured to accommodate medical articles of varying types and/or sizes. For example, when the medical article is a cannula, the medical securing device may be capable of accommodating and securing different cannula types, fluid lines and configurations.

The medical article securement device may be capable of securing a medical article to a body part of a patient easily, without training, and in a short period of time (e.g. less than 15 seconds).

The medical article securement device may be capable of being applied on top of existing dressings, such as adhesive sterile dressings (e.g. 3M Tegaderm™). The medical securing device may permit continuous visualisation of the medical article site and skin integrity.

At least a portion of the medical article securement device may be shaped to provide clearance for a dressing, such as adhesive sterile dressings. In embodiments in which the medical article is a needle free connector (e.g. for a cannula) at least a portion of the medical article securement device may be shaped to provide clearance for the wings of the needle free connector. For example, the base and/or the body engaging member may be trimmed or otherwise shaped in order to allow the cannula or needle free connector to flex or rotate about its axis to enable at least part of the wing and/or adhesive dressing to be disposed underneath the device. In some embodiments, the body engaging member and/or the base of the medical article securement device may present an elevated rim profile to provide clearance for a wing and/or adhesive dressing in use.

The medical securing device is a simple yet effective device for securing medical articles to a body part, and is suitable for high-volume low-cost mass manufacture.

In a third aspect there is provided a kit of parts for a medical article securement device according to the first aspect. The kit of parts may comprise:
a base having a top portion and a bottom portion, the top portion comprising a medical article engaging portion, optionally wherein the top portion and the bottom portion are separate parts that are configured to interconnect;
a securing arrangement for securing a medical article to the base;
a fastener provided in at least one of the base and/or the securing arrangement; and
a body engaging member, optionally wherein the body engaging member comprises a fastener for affixing the body engaging member around a body part.

The kit of parts may further include assembly instructions. The kit of parts may further include instructions for use.

In a fourth aspect there is provided a method of securing a medical article comprising:
providing a medical article securement device according to the first aspect;
approaching the medical article securement device to a medical article that has been located on a body part of a patient at the desired location;
guiding the medical article securement device under the medical article;
optionally rotating the medical article engaging member with respect to the body engaging member in order to align the medical article engaging portion with the medical article;
with the securing arrangement in the open configuration, engaging at least part of the medical article with the medical article engaging portion;
securing the medical article in the medical article engaging member by moving the securing arrangement to the closed configuration; and
securing the body engaging member of the medical article securement device to the body part of the patient.

The step of securing the medical article in the medical article engaging member by moving the securing arrangement to the closed configuration locks the locking mechanism and prevents rotation of the medical article engaging member relative to the body engaging member about the rotational axis of the base.

For the avoidance of doubt, locating the medical article on a body part of a patient at the desired location is not part of the method.

The medical article may be an invasive device comprising a conduit. The medical article may be selected from: a catheter, a cannula, a vascular access device, a lymphatic access device, a peripheral vascular access device, a central vascular access device, an arterial vascular access device, a venous vascular access device, a line, a midline, a peripherally inserted central catheter (PICC), a central arterial access device, a central venous access device, a central venous catheter (CVC), a subclavian line, an internal jugular line, a femoral line, a urinary catheter, a surgical drain.

The body part may be any body part, such as a torso, neck, limb, hand, paw, finger, toe, head, groin, armpit, reproductive organ, or the like. The body part may be a limb. The body part may be an arm or a leg. The body part may be located anywhere in the upper arm, the hand, the lower arm, the upper leg, the lower leg, or the foot. The body part may be a hand, a wrist, an elbow, a foot, an ankle, a calf, a thigh, a groin. The body part may be an upper arm. The body part engaging member may be wherein the medical article securement device is capable of being secured to both a left and a right limb. In other words, the medical article securement device may be ambidextrous. For example, the medical article securement device may be capable of being secured to both of the left arm and the right arm, the left hand and the right hand, the left leg and the right leg, or the left foot and the right foot.

### BRIEF DESCRIPTION OF THE FIGURES

FIG. 1 shows a perspective view of a medical article securement device according to an embodiment of the invention.

FIG. 2 shows an exploded view of the medical article securement device of FIG. 1.

FIG. 3 shows a medical article engaging member and a body engaging member of a medical article securement device according to another embodiment of the invention.

FIGS. 4A and 4B show exploded views of the medical article engaging member and a reinforcement portion of the body engaging member according to the medical article securement device of FIGS. 1 and 2.

FIGS. 5A and 5B show a perspective view and a front view of the top portion of the base of a medical article securement device according to some embodiments of the invention.

FIGS. 6A to 6C show vertical cross-sections of a medical article securement device according to some embodiments of the invention. FIG. 6B shows the device in the closed configuration (and without a medical article therein for clarity). FIG. 6A shows the device in the open configuration. FIG. 6C shows a schematic representation of the forces involved when the medical article securement device moves from the open configuration to the closed configuration. FIG. 6D shows a schematic representation of the medical article securement device according to an embodiment of the invention with a cannula, to illustrate the axes of the different portions of the medical article securement device and medical article in use.

FIGS. 7A and 7B, 8A and 8B and 9A show perspective views of medical article engaging members according to three embodiments of the invention. FIG. 9B shows a front view of the medical article engaging member of FIG. 9A.

FIGS. 10A-10D show the medical article securement device according to the invention secured to a hand and FIG. 10E shows the medical article securement device of FIGS. 10A-10D secured to a forearm.

FIG. 11A shows an exploded view of a portion of a medical article securement device according to another embodiment of the invention. FIGS. 11B and 11C show the same portion of the medical article securement device of FIG. 11A with the medical article engaging member assembled with the body engaging member and rotated in two different directions. FIG. 11D shows a perspective view of the complete medical article securement device of FIGS. 11A-11C.

FIGS. 12A and 12B show a portion of the medical article securement device according to another embodiment of the invention in the open and closed configurations respectively. FIGS. 12C and 12D show exploded views of the portion of the medical article securement device shown in FIGS. 12A and 12B. In this embodiment, the securing arrangement is a clamp.

FIGS. 13A and 13B show a portion of the medical article securement device according to another embodiment of the invention in the open and closed configurations respectively. FIGS. 13C and 13D show exploded views of the portion of the medical article securement device shown in FIGS. 13A and 13B. In this embodiment, the securing arrangement is a clamp similar to that shown in FIGS. 12A-12D but with a different locking mechanism.

FIGS. 14A and 14B show a portion of the medical article securement device according to another embodiment of the invention in the open and closed configurations respectively. FIGS. 14C and 14D show exploded views of the portion of the medical article securement device shown in FIGS. 14A and 14B. In this embodiment, the securing arrangement is a clamp similar to that shown in FIGS. 13A-13D but with a different locking mechanism.

FIGS. 15A and 15B show a portion of the medical article securement device according to another embodiment of the invention in the open and closed configurations respectively. FIGS. 15C and 15D show exploded views of the portion of the medical article securement device shown in FIGS. 15A and 15B. In this embodiment, the securing arrangement is a clamp similar to that shown in FIGS. 12A-12D and 14A-14D but with a different locking mechanism.

FIGS. 16A and 16B shows a portion of the medical article securement device according to another embodiment of the invention in the closed configuration. FIG. 16C shows an exploded view of the portion of the medical article securement device shown in FIG. 16A. In this embodiment, the securing arrangement is a clamp similar to that shown in FIGS. 15A-15D but with a slightly amended locking mechanism.

FIGS. 17A and 17B show renderings of a medical article securement device according to the invention. The securing arrangement in this embodiment is a clamp. In FIG. 1B the device is shown holding a smart site or needle free connector of a cannula.

FIG. 18 is an image of a patient with a rendering of the device of FIGS. 17A and 17B as it would be used to secure an IV in a medical setting.

FIG. 19A shows an exploded view of a medical article securement device according to another embodiment of the invention. In this embodiment, the securing arrangement is a strap which is pivotally connected to a locking element disposed between the base of the medical article engaging member and the body engaging member (or insert or base thereof). The locking element carries the second portion of the locking mechanism of the device, while the first portion of the locking mechanism is disposed on the base of the medical article engaging member.

FIGS. 19B and 19C shows side views of the device of FIG. 19A with a see-through detail of the locking mechanism of the device in the open configuration (when the securing arrangement is not tightened) and the closed configuration (when the securing arrangement is tightened) respectively.

FIG. 19D shows a cross-section of the device of FIGS. 19a, 19B, and 19C with a see-through detail of the annular snap arrangement between the base of the medical article engaging member and the body engaging member, as well as the engagement between the base of the medical article engaging member and the locking element.

FIG. 19 E shows a perspective view of the device of FIGS. 19A, 19B, and 19C, and 19D hosting a needle free connector (NFC) for a cannula and shows how the device is configured to accommodate NFCs with wings and/or dressings adhered thereto.

FIG. 19F shows perspective and side views of the device of FIGS. 19A, 19B, 19C, 19D and 19E hosting NFCs of different sizes.

FIG. 20A shows a perspective view of another medical article securement device according to another embodiment of the invention similar to the embodiment of FIGS. 19A-F. In this embodiment, the securing arrangement is also a strap with apertures configured to be secured to a protrusion of the base.

FIGS. 20B and 20C show top and side perspective views of the device of FIG. 20A with a see-through detail of the locking mechanism.

FIGS. 21A and 21B show perspective views of the locking mechanism of a medical article securement device according to another embodiment of the invention in the open and closed configurations respectively.

FIG. 22A shows a perspective view of three embodiments 1930A, 1930, and 1930C of the securing arrangement.

FIG. 22 B shows a perspective view of a medical article securement device according to another embodiment of the invention.

FIG. 22C shows an exploded view of the medical article securement device 1900 of FIG. 22B with any one of the securing arrangement straps 1930A, 1930B, or 1930C of FIG. 22A.

### DETAILED DESCRIPTION OF THE FIGURES

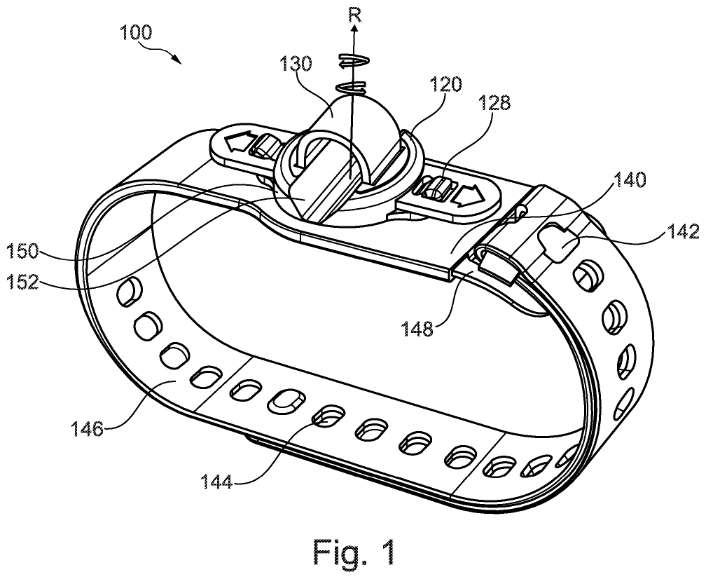

FIG. 1 shows a perspective view of a medical article securement device 100 according to an embodiment of the invention. The device 100 comprises a medical article engaging member 150 comprising:
a base 120 defining at least one medical article engaging portion 152 (or holder) configured to receive a portion of a medical article, and
a securing arrangement 130 for reversibly securing the medical article to the base 120, the securing arrangement 130 defining an open configuration and a closed configuration;
a body engaging member 140 configured to affix the medical article securement device 100 to a body part of a human or animal; and
a locking mechanism (not visible in this figure) for the medical article engaging member 150 and the body engaging member 140. In this embodiment, the locking mechanism is configured to allow rotation of the medical article engaging member 150 (base 120 and securing arrangement 130) relative to the body engaging member 140 about a rotational axis R of the base 120 when the securing arrangement 130 of the medical article engaging member 150 is in the open configuration. The locking mechanism is configured to prevent rotation of the base 120 of the medical article engaging member 150 relative to the body engaging member 140 about the rotational axis R of the base 120 when the securing arrangement 130 of the medical article engaging member 150 is in the closed configuration.

In this embodiment, the body engaging member 140 is a strap 146 configured to be disposed around a body part of a human or an animal and be secured to the body part by means of a fastener 142, which in this embodiment is a buckle configured to engage one of the apertures 144 of the strap.

In this embodiment, the strap 146 of the body engaging member 140 is made of a resilient material, (e.g. silicone), which in use provides a maximum friction against the skin of a patient to minimise micromovements of the device 100 during use and secure the device 100 in the correct location on the body part. The strap 146 defines a plurality of apertures 146 which double up as the holes for the fastener 142 (functioning as buckle locations which can hook in the buckle, thus enabling the device to be secured around body parts of different sizes) and also as ventilation apertures to minimise accumulation of perspiration (sweat) when a patient wears the device 100.

The body engaging member 140 comprises a reinforced segment or insert 148, which is resistant to stretching. The reinforced segment 148 is moulded from any material that is sufficiently flexible to be curved around a portion of a patient's limb or other body part when the device is attached thereto, but which is substantially undeformed under the action of the lateral forces applied between the straps, in use. For example, the reinforcement segment may be moulded from polypropylene. The reinforcement segment may not stretch as the strap (e.g. silicone strap) 146 does. The reinforcement segment 148 may be configured to be encapsulated within the silicone strap 146 and therefore, in use, the reinforced segment 148 may not be in contact with a patient's skin. Advantageously, the reinforced segment 148 may prevent a 360° tourniquet effect (i.e. preventing blood flow) on the body part of the wearer to which the medical article securement device is attached in use.

The medical article engaging portion 150 is capable of rotating 360° with respect to the body engaging portion 140 in the open configuration. The rotation may occur about the rotational axis R of the base 120, and it may be in a clockwise and/or an anti-clockwise direction. Advantageously, this 360° rotation may accommodate infinite medical article orientations as well as the use of the device 100 on left or right limbs. The medical article engaging portion 150 may rotate in predetermined intervals, such as in 5° to 45° intervals, such as in 5°, or 10°, or 15°, or 20°, or 25°, or 30°, or 35°, or 40°, or 45° intervals. In other words, the rotation of the medical article engaging portion may be indexed. Advantageously, indexing the rotation of the medical article engaging member 150 enables precise and quick fine tuning of the position of the medical article engaging member 150.

Rotation of the medical article engaging member with respect to the body engaging member may provide aural feedback, such as audible and/or tactile feedback, such as a clicking sound and/or a tactile click which may reassure a user that the device is being secured correctly. The aural feedback may be generated every time the medical article engaging portion rotates a predetermined interval or distance as defined above. The aural feedback may be achieved by providing an indexer (e.g. a spring loaded or resilient indexing tab or pole) on the medical article engaging portion (e.g. on the base) such that the indexer moves over a toothed feature (e.g. toothed ring) disposed on the body engaging portion or on an insert to which the body engaging portion is attached. Upon rotation of the medical article engaging portion with respect to the body engaging portion (or insert thereof), the indexer may go over the profile of every tooth and after passing the crest of the tooth move to the gullet of the next tooth providing a clicking sound and feel.

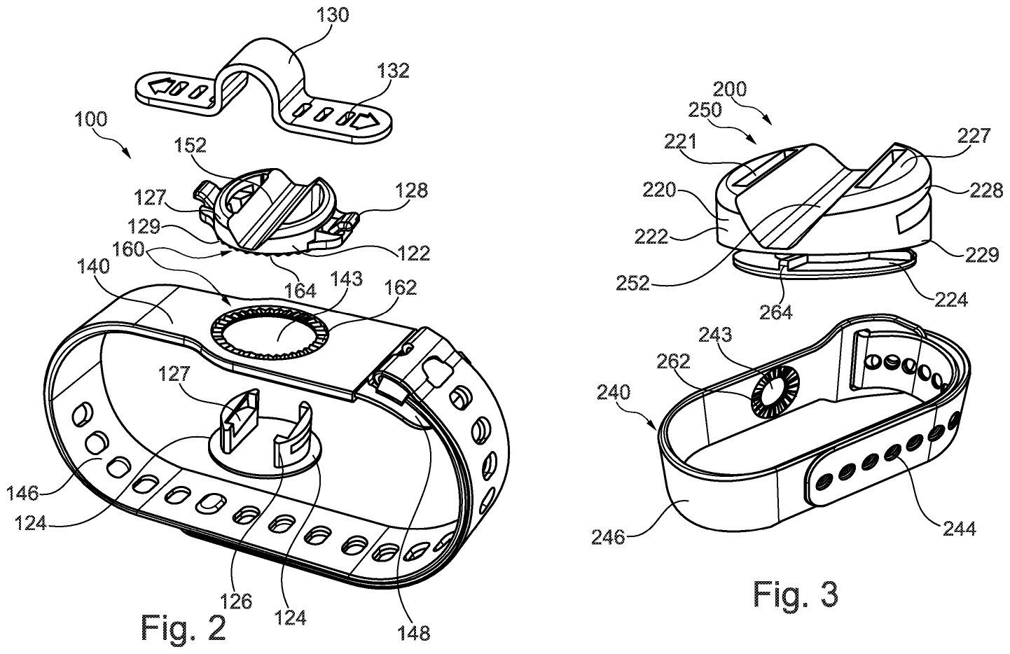

As better shown in FIG. 2, which shows an exploded view of the device 100, the medical article engaging member 150 comprises a base 120 which has a top portion 122 and a bottom portion 124, which in this embodiment are separate parts. The body engaging member 140 (the strap 146 and the reinforcing segment 148 thereof) is sandwiched between the top portion 122 and the bottom portion 124 of the base 120. The bottom portion 124 comprises two protrusions 126 configured to engage the top portion 122 of the base above the upper surface of the body engaging member 140. The protrusions 126 are fed through a aperture 143 defined in the strap 146 and the reinforcing segment 148.

The top portion 122 of the base defines two apertures 123, each configured to receive the corresponding projection 126 of the bottom portion 124 of the base 120.

The base 120 may comprise a hard material, such as moulded plastic, and/or a resilient material, such as an elastomeric material, synthetic rubber, natural rubber, medical grade silicone. The top portion 122 of the base 120 may comprise a hard material. The hard material may be lined (at least in the medical article engaging portion 152) with a soft or resilient material at the medical article engaging portion. For example, the top portion of the base may comprise hard plastics material lined with elastomeric material or silicone. The lining material may be configured to provide friction against the medical article in order to prevent micromovements, as well as to accommodate medical articles of different shapes and/or sizes. Alternatively, the top portion may be moulded in a resilient material, such as rubber, silicone or the like. Advantageously, providing a medical article engaging portion made of or lined with a resilient material or a soft material such as silicone or rubber may maximise the friction coefficient of the material configured to be in contact with a medical article, thus maximising the friction force exerted by the medical article engaging portion on the medical article, and further assisting in the clamping and securement of the medical article within the device to prevent longitudinal, lateral and/or rotational movement of the medical article within the device.

The securing arrangement 130 in this embodiment is a pull band or strap made of a resilient material (e.g. rubber or silicone) and defining apertures 132 configured to be hooked on a hook or wing 128 so that the securing arrangement 130 can be secured to the base 120 at different lengths. In other words, the securing arrangement 130 is capable of being tightened and loosened (e.g. by the fastener) at different predetermined lengths (defined by the separation between apertures 132). The predetermined length may be from 0.5 mm to about 15 mm, preferably from about 10 mm to about 14 mm. Advantageously, providing a securing arrangement that may be fastened at different lengths enables the medical article securement device to be used with medical articles of different sizes (e.g. cannulas of different diameters), and therefore be universal.

Advantageously, the resilient material of the securing arrangement 130 has a dampening or shock absorbing effect on any force exerted on a medical article which (in use) is secured in the medical article securement device 100. Additionally, the resilient material maximises the friction coefficient of the material of the securing arrangement, thus maximising the friction force exerted by the securing arrangement 130 on the medical article, thus further assisting in the clamping and securement of the medical article within the device 100 to prevent longitudinal, lateral and/or rotational movement of the medical article within the device 100.

In the open configuration, the securing arrangement 130 is configured to engage the base 120 so as to allow movement of the securing arrangement 130 relative to the base 120 along a longitudinal axis of the securing arrangement 130. Movement of the securing arrangement 130 relative to the base 120 along the longitudinal axis of the securing arrangement 130 is configured to modify the distance between the medical article engaging portion 152 of the base 120 and a section of the securing arrangement 130 disposed opposite the medical article engaging portion of the base. Specifically, pulling from the pull band 130 away from the base 120 on one of both ends of the pull band 130 when the band is fed through the slits or channels 221 of the base, tightens the pull band 130 towards the medical article engaging portion 152, which in this embodiment is a V-shaped channel. Once the desired tension is achieved (when the device receives a medical article, such as a cannula), then the pull band 130 is secured by hooking the aperture 132 which provides the desired tension on the hook 128 of the base. When the medical article needs to be removed or when the setting needs to be adjusted, the pull band 130 can be pulled away from the base again and released from the hook 128, thus moving the device 100 to the open configuration.

The locking mechanism 160 in this embodiment comprises a first lock component 162 on the medical article engaging member (teeth disposed on a lower surface 129 of the top portion 122 of the base) and a second lock component 164 on the body engaging member 140 (corresponding teeth 164 defined on the reinforced segment 148 and which are configured to interact with the teeth 162 of the top portion 122 of the base through the aperture 143 of the body engaging member 140. The distance between the first lock component (teeth 162) and the second lock component (teeth 164) is configured to be greater when the securing arrangement (pull band 130) is in the open configuration than when the securing arrangement 130 is in the closed configuration.

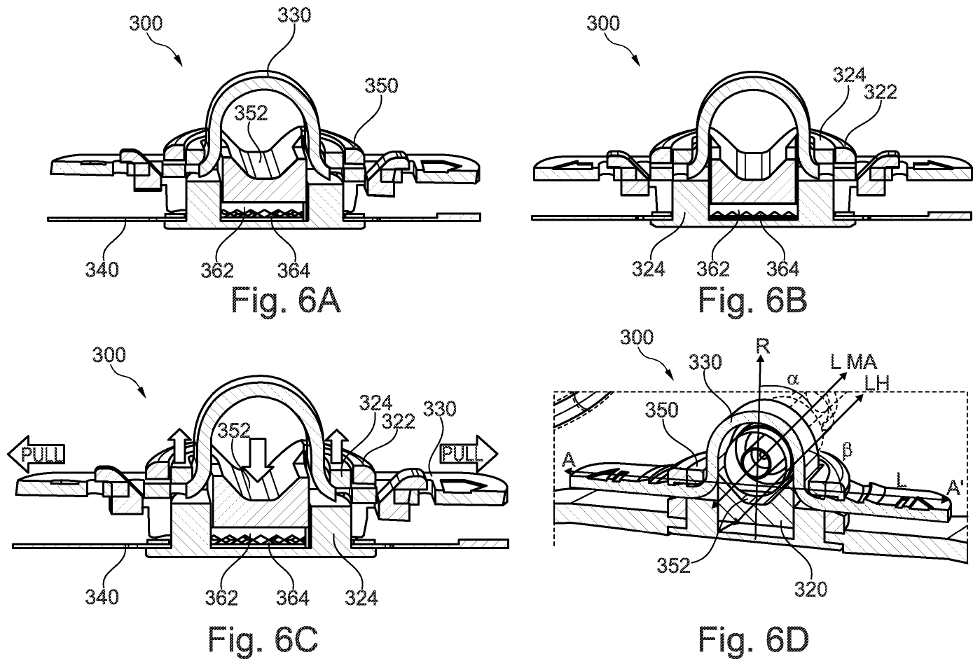

FIGS. 6A to 6C show vertical cross-sections of a medical article securement device according to some embodiments of the invention. FIG. 6B shows the device in the closed configuration (and without a medical article therein for clarity). FIG. 6A shows the device in the open configuration. Features of the securement device in common with the securement device 100 are provided with like reference numerals, incremented by 200.

This the change in distance between the first lock component 364 and the second lock component 362 is better observed in FIG. 6B, where it is visible that the teeth 362 engage the teeth 364 and provide a frictional and mechanical lock that prevents rotation of the medical article engaging member 350 with respect to the body engaging member 140. In this embodiment, the distance between the top portion 322 of the base and the bottom portion 324 of the base also decreases in the closed configuration and therefore the body engaging member which is sandwiched between the top portion 322 and the bottom portion 324 of the base is squeezed in more tightly and also assists in the frictional engagement of the base with the body engaging portion 340, thus further preventing rotation of the base.

In contrast, in FIG. 6A it is shown that in the open configuration (e.g. when the securing arrangement 330 is looser) the distance between the teeth 362, 364 is large enough for the teeth to be disengaged and allow rotation of the medical article engaging member 350 with respect to the body engaging member 340.

FIG. 6C is a schematic representation of the forces that are involved in the device 300 when moving from the open configuration to the closed configuration with a medical article in the medical article receiving portion (the medical article is not shown in the device for a better representation of the forces involved). When pulling the securing arrangement 330 away from the base, the securing arrangement subjects the bottom portion 324 of the base to a pulling force away from the body engaging portion 340 and simultaneously exerts a pushing force (downwards in this image) on the top portion 322 of the base. The body engaging member 340 is clamped between the top portion 322 and the bottom portion 324 of the base, thus forcing the teeth of the first lock component 362 and the second lock component 364 to engage, thus preventing further rotation of the base. In the closed configuration, the securing arrangement 330 subjects a medical article (e.g. a luer lock of a cannula) disposed on the medical article engaging portion 352 to a pushing force towards the base.

These opposing forces prevent longitudinal, rotational and/or lateral movement of the cannula engaged within the medical article engaging portion (i.e. the holder). Advantageously, the medical engaging member acts as a lock, or a pincer, clamping the cannula between the base 320 and the securing arrangement 330, and thus preventing displacement of the cannula or rotation of the cannula about its longitudinal axis L. This is particularly advantageous as the securing arrangement 330 and the base (or medical article engaging portion 352 thereof) together are configured to counteract any force applied on the cannula (e.g. a tugging force, or a rotational force that is often required to be applied in a human or animal healthcare setting, for example to change or disconnect the tubing connected to a luer lock of the cannula).

FIG. 6D depicts the longitudinal axis L MA of a medical article (a cannula in this embodiment), which is parallel to the longitudinal axis L H of the medical article engaging portion or holder. Both L MA and L H are disposed at an angle α of from about 55° to about 90° with respect to the rotational axis R of the base 320 and at an angle β of from about 0° to about 35° with respect to the longitudinal axis L of the securing arrangement 330, which is defined between the ends A, A′ of the securing arrangement 330. In this embodiment, the top portion 320 of the base may be moulded in a resilient material such as silicone.

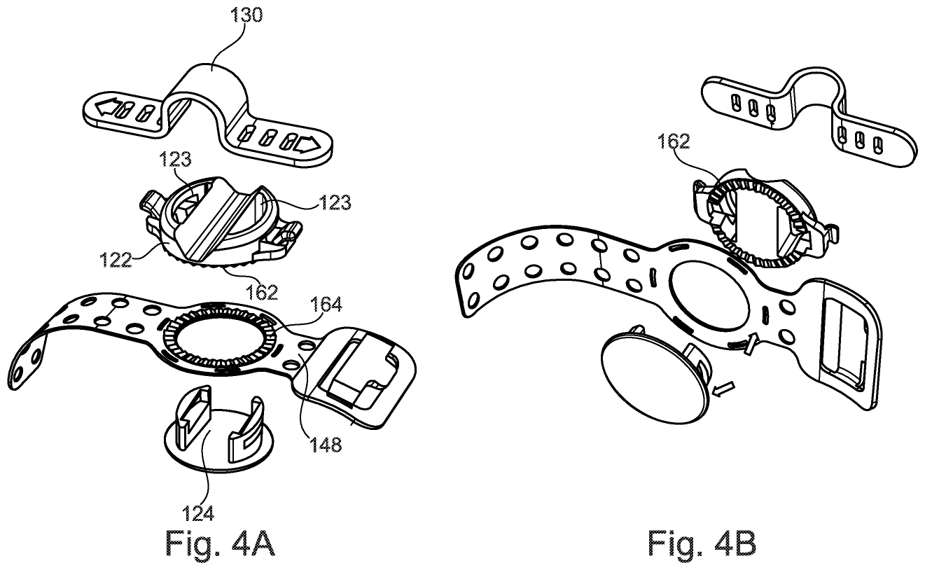

The locking mechanism 160 of the device 100 can be better observed in FIGS. 4B and 4A, which clearly show teeth 164 of the reinforced segment 148 of the body engaging member 140 (for ease of visualisation of the locking mechanism the strap 142 has been omitted in this drawing) and the corresponding teeth 162 of the top portion 122 of the base. As shown by the arrows in FIG. 4B, the locking mechanism could also be disposed between a lower surface of the body engaging member 140 (e.g. on a lower surface of the reinforcing segment 148) and an upper surface of the bottom portion 124 of the base.

Also shown clearly in FIG. 4A are two slits or channels 123 defined in the top portion 122 of the base. The securing arrangement 130 (pull band) is configured to be fed through these channels 123 and slide through the channels 123 while moving allow along a longitudinal axis of the securing arrangement 130 to tighten and/or release the securing arrangement 130. FIG. 1 shows that each end of the securing arrangement 130 has been fed through one of the channels 123 and subsequently the securing arrangement has been moved to the closed configuration by hooking the holes 132 of the securing arrangement 130 to the hooks or wings 128 of the base.

FIG. 3 shows a medical article engaging member 250 and a body engaging member 240 of a medical article securement device 200 according to another embodiment of the invention. Features of the securement device in common with the securement device 100 are provided with like reference numerals, incremented by 100. In this embodiment, the base 220 is made of one piece and comprises a top portion 222 and a bottom portion 224 and defines a groove or recess 226 therebetween. The recess 226 is configured to be located in the aperture 243 of the strap 246 of the body engaging member, with the bottom portion 224 of the base 220 disposed on the lower surface of the strap 246 and the top portion 222 disposed on the upper surface of the strap.

In this embodiment, the locking mechanism comprises a series of teeth 262 defined on the lower surface of the strap 246 and indexers or protrusions 264 defined on an upper surface of the bottom portion 224 of the base 220. In use, the indexers 264 engage the teeth 262 of the strap 246 and allow rotation of the medical article engaging member 250 in the open configuration but not in the closed configuration. For the avoidance of doubt, the lower surface of the strap is configured to face towards the body part of the wearer to which the device 200 is attached in use.

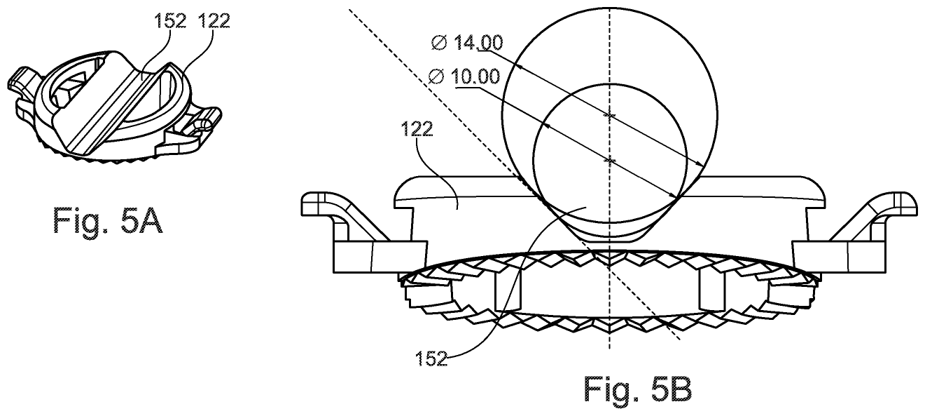

In both the medical article engaging member 150 and 250 of FIGS. 2 and 3, the medical article engaging portion 152 is a V-shaped channel defined on the top surface 127, 227 of the top portion of the base. The channel 152 is disposed at an angle with respect to the lower surface 129, 229 of the top portion, with respect to the bottom portion of the base and with respect to the body engaging portion 140, 240. The angle may be from about 0° to about 35°, preferably about 7°. The tapering nature of the channel can be better observed in FIG. 5A. Providing a slanted and tapered channel for receiving the medical article is optimal as it counteracts the tendency of invasive medical articles (such as transcutaneal medical articles, for example cannulas) to pull out or fall due to the weight of the medical article and the angle of insertion of the medical article into the patient.

Providing a channel, and preferably a V-shaped channel as the medical article engaging portion, may enable universal use of the medical article securement device with medical articles of different sizes as the channel may be capable of wedging or securely hosting the medical articles irrespective of their size. For example, when the medical article is a cannula, the medical article securement device may be able to secure cannulas of different diameters, and therefore be universal. This is better observed in the schematic representation of FIG. 5B. In FIG. 5B, it can be seen that two medical articles with a 40% difference in diameter can independently be located in the same medical article engaging member.

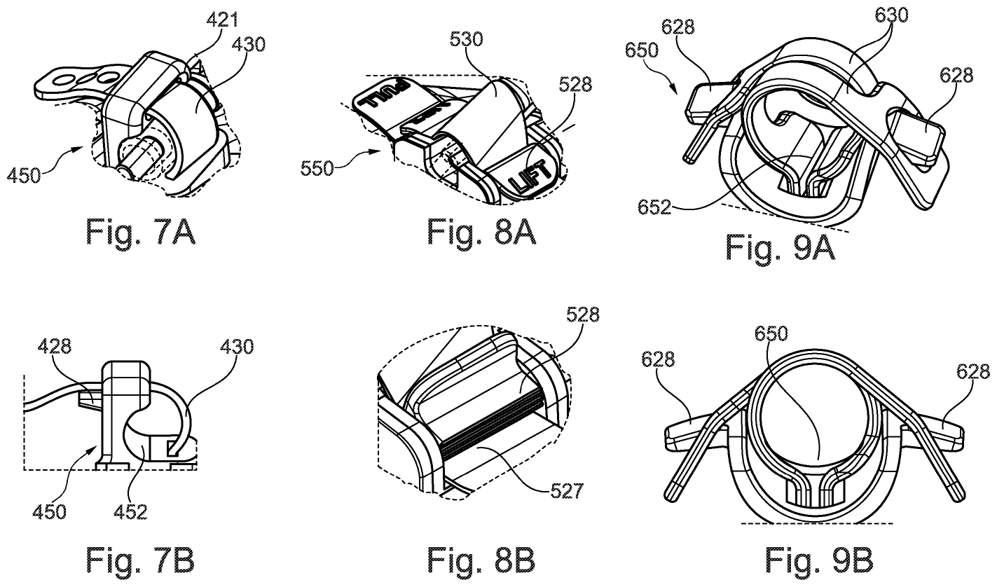

FIG. 7A shows a perspective view of a medical article engaging member 450 according to another embodiment of the invention. FIG. 7B, shows a front view of the medical article engaging member 450 of FIG. 7A. Features of the medical article engaging portion 450 in common with the medical article engaging portion 150 are provided with like reference numerals, incremented by 300.

The securing arrangement 430 is a flexible band (e.g. made of silicon), which is fixedly or pivotally coupled to the base at one side of the medical article engaging portion 452. The base defines a securing arrangement slit or channel 421 at the opposite side of the medical article engaging portion 452. In use, a medical article (a luer lock of a cannula is depicted in FIG. 7A) rests against the medical article engaging portion 452 and the securing arrangement 430 is disposed over and around the luer lock of the cannula to prevent dislodgement of the medical article from the medical article engaging portion or movement therein. Once the medical article is located in the correct place and the securing arrangement is tightened, the securing arrangement is configured to be fastened with fastener 428 to minimise or prevent movement of the medical article with respect to the medical article engaging portion 452.

FIG. 8A shows a perspective view of a medical article engaging member 550 according another embodiment of the invention. FIG. 8B shows an enlarged view of the fastener 528 of the securing arrangement 530 in the open configuration. Features of the medical article engaging member 550 in common with the medical article engaging member 150 are provided with like reference numerals, incremented by 400. In this embodiment the base defines two slits or medical securing arrangement slits or channels 521 disposed at either side of the medical article engaging portion. The medical securing arrangement 530 slides through the slits 521 and is tightened around a cannula received in the device. In order to secure the securing arrangement, the fasteners 528 are pushed down to trap the securing arrangement 530 and prevent movement of the cannula within the device.

FIG. 9A shows a perspective view of a medical article engaging member 650 according another embodiment of the invention. FIG. 9B shows a front view of the medical article engaging member of FIG. 9A. Features of the securement device in common with the securement device 100 are provided with like reference numerals, incremented by 500. In this embodiment, the securing arrangement 630 comprises two bands (e.g. made of silicone) which are fixed at the base to form the medical article receiving portion 652 as a V-shaped channel. Each of the straps 630 is configured to wrap over and around a medical article from an opposite side and be secured on a hook 628 formed on the base at either side of the medical article engaging portion 652.

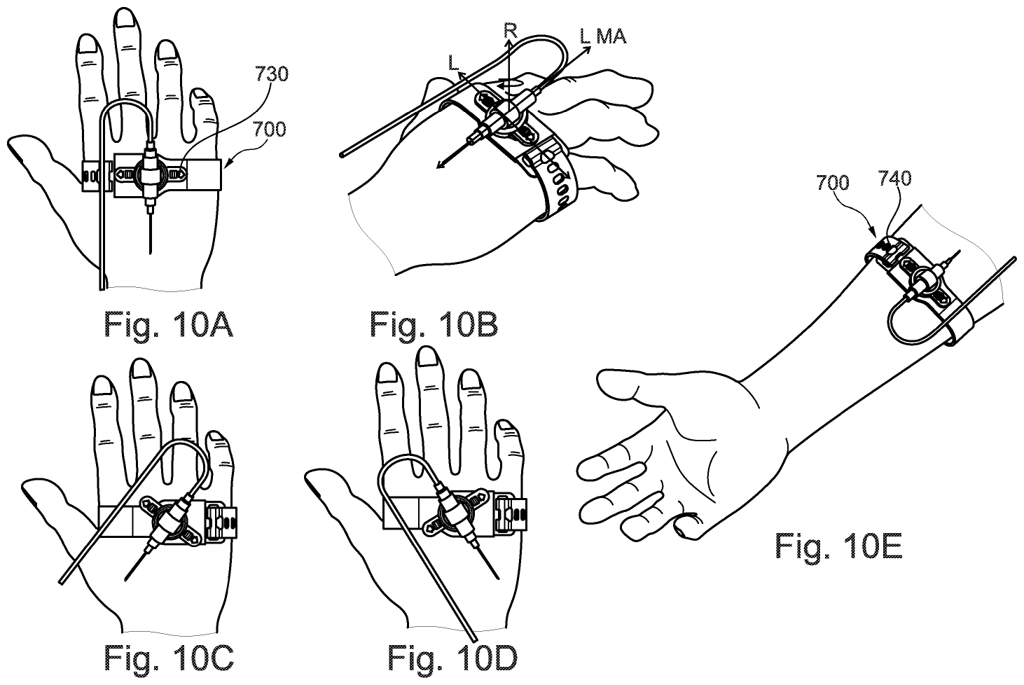

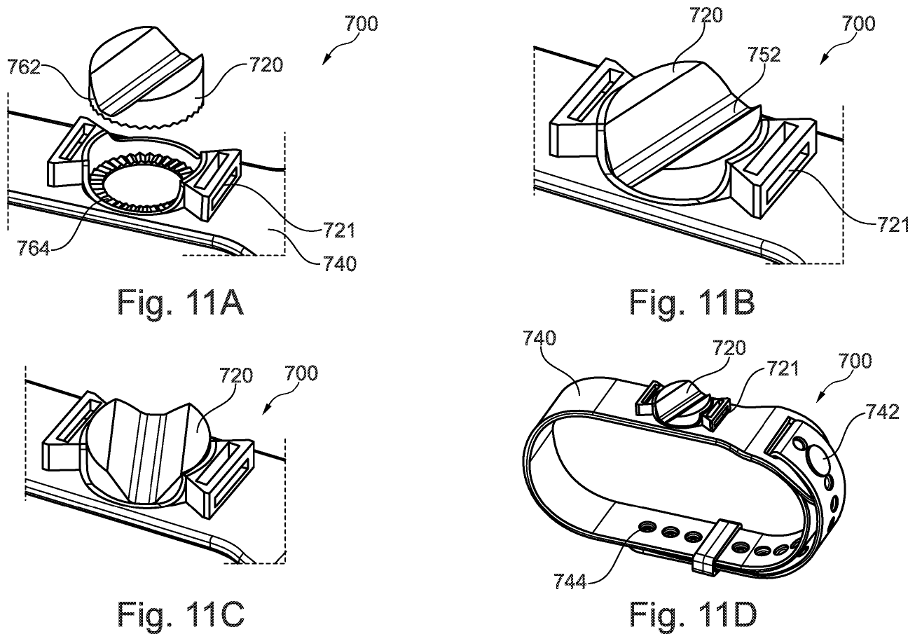

FIGS. 10A-10D show the medical article securement device 100 of FIG. 1 secured to a hand and FIG. 10E shows the medical article securement device of FIGS. 10A-10D secured to a forearm. In FIGS. 10A, 10B and 10E the longitudinal axis L of the securing arrangement 730 is aligned with the longitudinal axis of the body engaging member so that a medical article (e.g. a cannula) can be secured when it is inserted straight (i.e. with its longitudinal axis L MA disposed along the direction of an arm of a patient). FIGS. 10C and 10D show that the medical article engaging member 550 may be rotated about a rotational axis R of the base 520 in a clockwise or anti-clockwise direction in order to accommodate any angle of insertion of the cannula into a patient, as well as enable use of the device on left or right limbs of a patient. The body engaging member can be adjusted to fit the larger diameter of the forearm, as well as the smaller diameter of the arm of a patient.

FIG. 11A shows an exploded view of a portion of a medical article securement device 700 according to another embodiment of the invention. Features of the securement device in common with the securement device 100 are provided with like reference numerals, incremented by 600. In this embodiment, the base 720 of the medical article engaging member only comprises a top portion which in use is located on the upper surface of the body engaging member 740 (as seen in FIGS. 11B-11D). The base 720 defines a series of teeth 762 on its lower surface and a medical article engaging portion 752 on its upper surface. The medical article engaging portion is a V-shaped channel as in other embodiments described herein and it will not be described in further detail. The body engaging portion 740 defines a corresponding set of teeth 764 configured to engage teeth 762 in the closed configuration. The body engaging portion also defines protrusions at opposing sides of the locking mechanism. Each protrusion defines a slit or channel 721, which is configured to receive a securing arrangement (not shown) and act as the fastener for the securing arrangement.

As better seen in FIGS. 11B and 11C, the base 720 is capable of rotating clockwise and anticlockwise so that an end-user (e.g. a nurse, nursing assistant, vet or doctor) can align the medical article engaging portion 752 with a medical article (e.g. a cannula) which has been previously located or inserted in a patient. Once the longitudinal axis of the medical article receiving portion 752 and the medical article (not shown) have been aligned, the medical article may be inserted in the medical article receiving portion 752 of the base 720 and the securing arrangement may be fed through the slits 721 of the body engaging member 740 and tightened. Tightening the securing arrangement around the medical article exerts a downward force on the medical article towards the base 720 and simultaneously an upward force on the body engaging member 740 (or protrusions thereof), which causes the locking teeth 764 and 762 to engage and actuate the locking mechanism, thus preventing any further rotation of the base 720 with respect to the body engaging portion 740.

FIG. 11D shows a perspective view of the complete medical article securement device of FIGS. 11A-11C. In this figure it can be observed that the body engaging portion also comprises a fastener having a popper 742 and multiple apertures 744 which can be engaged in the popper 742 in order to provide an adjustable body engaging member which can fit most children and adult body parts.

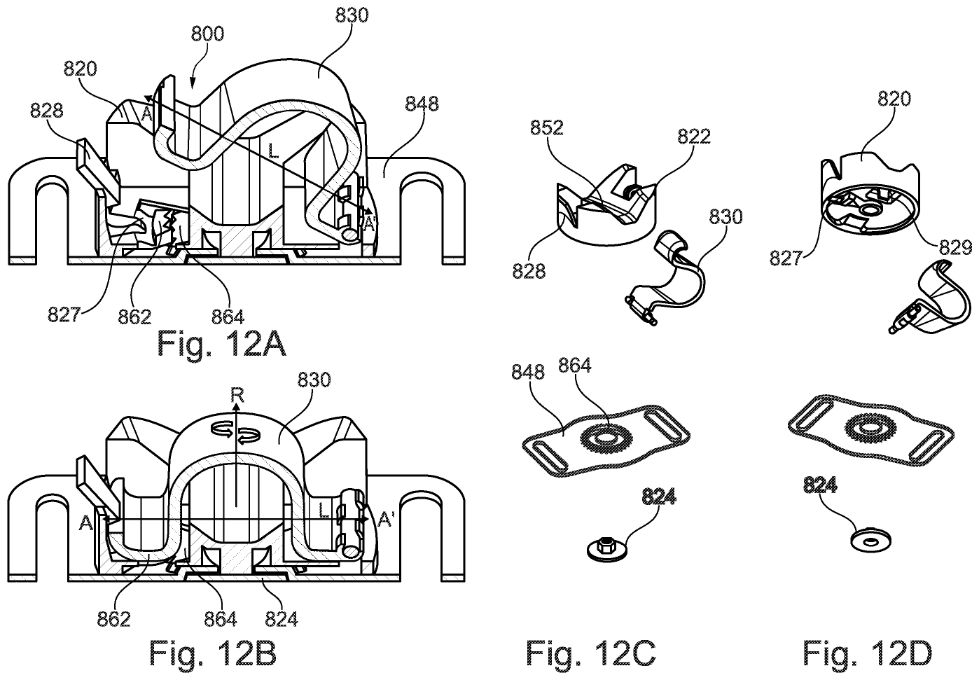

FIGS. 12A and 12B show a portion of the medical article securement device according to another embodiment of the invention in the open and closed configurations respectively. FIGS. 12C and 12D show exploded views of the portion of the medical article securement device shown in FIGS. 12A and 12B. In this embodiment, the securing arrangement is a clamp. Features of the securement device in common with the securement device 100 are provided with like reference numerals, incremented by 700.

These figures show medical article securement device 800 comprising:
a medical article engaging member 850 comprising:
   a base 820 defining at least one medical article engaging portion 852 configured to receive a portion of a medical article (including but not limited to a cannula, a needle free connector or a fluid line connector), and
a securing arrangement, which in this embodiment is a clamp 830 fabricated of a rigid material (e.g. plastic) for reversibly securing the medical article to the base 820. One end of A′ the clamp 830 is pivotally coupled to the base 820 at one side of the medical article engaging portion (channel for receiving the medical article). The clamp 830 defines an open configuration (FIG. 12A) in which the end A of the clamp which is not pivotally coupled to the base 820 is not engaged with the base 820, and a closed configuration (FIG. 12B) in which the end A of the clamp 830 reversibly engages with the base 820 at an engagement site, which in this case is a leaver, latch or tab 828 which provides a quick set (or snap-fit) closure for the clamp and enables a quick release if an end-user deflects the leaver 828 so that end A of the clamp 830 can be freed. Engagement site 828 is spring loaded and biased towards the position in which the clamp cannot be freed when in the closed configuration. The longitudinal axis L of the clamp 830 is defined between the ends A-A′ of the clamp. As seen in FIG. 12B, the longitudinal axis L of the clamp 830 is substantially perpendicular to the rotational axis R of the base when the securing arrangement is in the closed configuration (FIG. 12B). Therefore, the clamp 830 is a quick release/quick set securing arrangement.

In these figures there is only shown an insert or base 848 of the body engaging member which is configured to affix the device 800 to a body part of a human or animal. The device 800 has a locking mechanism 862, 864 for the medical article engaging member and the body engaging member respectively. The base 820 comprises the first lock component 827 disposed at a distance from the second lock component 864, which is a toothed ring disposed on the rigid insert or base 848 of the body engaging member. The first lock component is 827 a resilient tab or cantilever arm with teeth 862. The tab 827 extends substantially parallel to the insert/base 848 of the body engaging member and at a close distance from the second lock component 864, but allowing free rotation of the base 820 with respect from the body engaging member (and insert/base 848 thereof) when the clamp is in the open configuration (FIG. 12A). The second lock component 864 is a toothed feature (ring) configured to engage with the toothed feature 862 of the first lock component. The insert or base 848 of the body engaging member comprises slots or holes through which the body engaging member (e.g. a strap) may be fed for securing the body engaging member to the insert or base 848. Starting from the open configuration (FIG. 12A), pressing on the clamp 830 subjects the clamp to pivotal movement and the end of the clamp A which is not pivotally coupled to the base may press against the first lock component 827/862 and deflect it until the teeth 862 of the first lock component engage with the teeth of the second lock component 864. The clamp may be secured in the closed configuration by the leaver, latch or tab 828. In the closed configuration, a medical article disposed in the medical article engaging portion is pinched between the clamp and the medical article engaging portion and thus prevented from lateral and/or rotational movements. Simultaneously, moving the clamp 830 to the closed configuration, actuates the locking mechanism and prevents rotation of the medical article engaging member with respect to the body engaging member. To move the clamp to the open position, latch (or resilient tab) is deflected to provide clearance for the clamp to pivot back to the open configuration. This in turn causes the resilient tab (or cantilever arm) to spring back to its original position which is not in engagement with the teeth of the second lock component, thus allowing the medical article engaging member to rotate again.

FIG. 12 D best shows that the base 820 also has an indexer tab 829 which is configured to be in contact with the teeth of the toothed wheel 864 of the second lock component of the body engaging member insert/base 848. Upon rotation of the base with respect to the body engaging member, the indexer tab will brush against teeth 864 and produce a clicking feel and sound.

FIGS. 12D and 12C show that base 820 has a top portion 822 and a bottom portion 824, and the insert of the body engaging member 848 is configured to be sandwiched between the top portion 822 and the bottom portion 824.

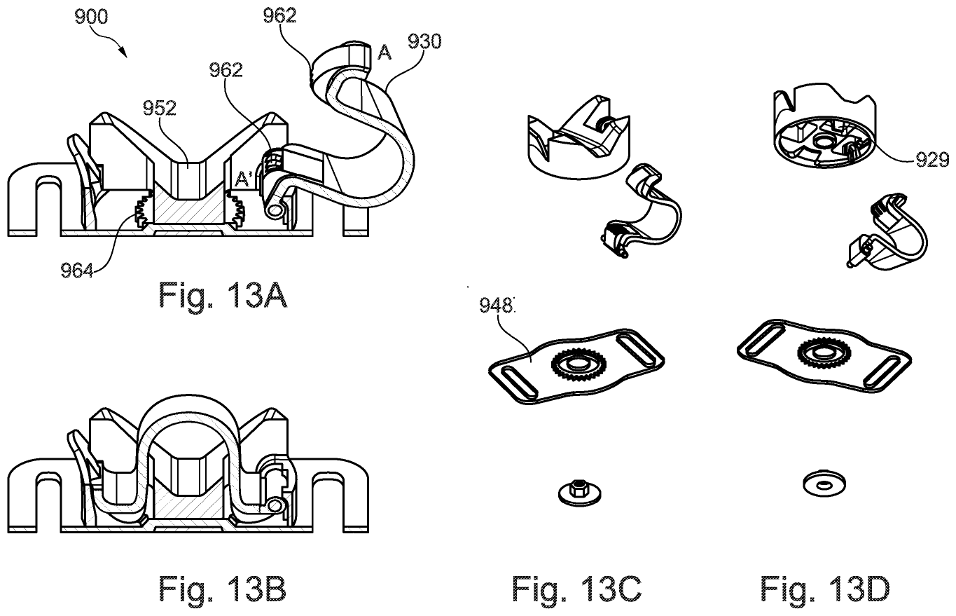

FIGS. 13A and 13B show a portion of the medical article securement device 900 according to another embodiment of the invention in the open and closed configurations respectively. FIGS. 13C and 13D show exploded views of the portion of the medical article securement device shown in FIGS. 13A and 13B. In this embodiment, the securing arrangement is a clamp similar to that shown in FIGS. 12A-12D but with a different locking mechanism. Features of the securement device in common with the securement device 100 are provided with like reference numerals, incremented by 800. The features in common with the device of FIGS. 12A-12D will not be discussed in detail as the principle of operation and general construction is the same.

The clamp of FIGS. 13A-D works in a similar manner to that of FIGS. 12A-D but the locking mechanism is different as first lock component 962 are teeth provided on both ends A, A of the clamp 930, and the second lock component remains the toothed ring 964 disposed on the insert/base 948 of the body engaging member. Since the clamp comprises toothed features disposed in two locations which, in the closed configuration (FIG. 13B) are disposed at either side of the medical article engaging portion 952, this device experiences opposing engagement forces of the first lock components 962 with the second lock component 964 when the clamp is in the closed configuration. As the clamp starts to pivot towards the closed configuration, the first set of teeth 1062 will engage with the toothed ring 964 starting the rotational locking action. When the clamp is further pushed until end A engages at the engagement site with the leaver 928, the second set of teeth 962 will also engage with the toothed ring 964 at a diametrically opposed location. Therefore, this arrangement may provide a stronger lock that is less likely to fail under strain. As seen in FIG. 13D, the base also defines indexer 929 to provide an indexed rotation with aural indication that rotation is taking place.

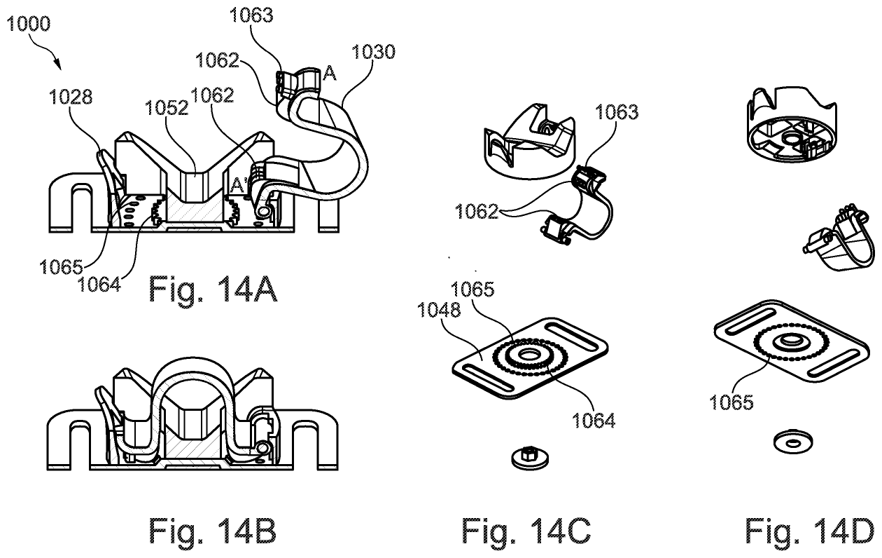

FIGS. 14A and 14B show a portion of the medical article securement device 1000 according to another embodiment of the invention in the open and closed configurations respectively. FIGS. 14C and 14D show exploded views of the portion of the medical article securement device shown in FIGS. 14A and 14B. In this embodiment, the securing arrangement is a clamp similar to that shown in FIGS. 13A-13D but with a different locking mechanism. Features of the securement device in common with the securement device 100 are provided with like reference numerals, incremented by 900. The features in common with the device of FIGS. 13A-13D will not be discussed in detail as the principle of operation and general construction is the same.

Device 1000 has the same construction as device 900, with toothed features 1062 disposed on a lower surface of both ends A, A′ of the clamp 1030, said toothed features 1062 configured to engage with the corresponding teeth of the toothed ring 1064 of the body engaging member insert. In addition, the free end A of the clamp 1030 comprises a series of male protrusions (pegs) 1063 which engage with a series of apertures 1065 defined on the insert 1048 of the body engaging member. The addition of a third lock in the form of mating male/female features 1063/1065 of the clamp 1030 and the insert 1048 of the body engaging member allows for stronger engagement between the first and second lock components and therefore creates a stronger lock.

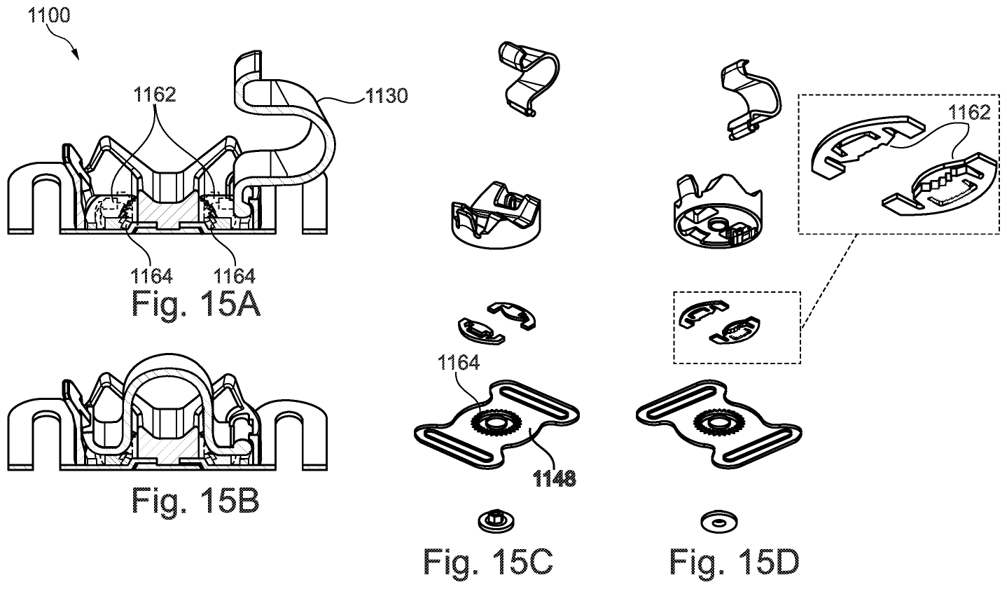

FIGS. 15A and 15B show a portion of the medical article securement device 1100 according to another embodiment of the invention in the open and closed configurations respectively. FIGS. 15C and 15D show exploded views of the portion of the medical article securement device shown in FIGS. 15A and 15B. In this embodiment, the securing arrangement is a clamp similar to that shown in FIGS. 12A-12D and 14A-14D but with a different locking mechanism. Features of the securement device in common with the securement device 100 are provided with like reference numerals, incremented by 1000.

The clamp of the embodiments of FIGS. 15A-D does not bear the first lock component. Instead, the first lock component 1162 is provided on two locking inserts located between the clamp 1130 and the insert of the body engaging member 1148 at diametrically opposed locations. The locking insert 1162 comprises a cantilevered tab comprising a toothed feature or profile. The second lock component 1164 is a toothed ring disposed on the insert 1148 of the body engaging member and configured to engage with the toothed features of the first lock component 1162 at diametrically opposed locations. The toothed features of the first lock component 1162 and the second lock component 1164 may be angled or tapered and complementary to each other.

In some embodiments, in the open configuration, the toothed feature of the first lock component 1162 may be configured to engage with the toothed feature of the second lock component 1164. Subjecting the medical article engaging member to a rotational force in the open configuration may force a deflection of the cantilevered tabs 1162 and may allow rotation with a clicking sound. Pressing on the clamp 1130 may subject the clamp to pivotal movement and the ends of the clamp may press against the locking insert(s) and deflect it/them until the teeth of the first lock component 1162 intimately engage with the teeth of the second lock 1164 component (FIG. 15B). Advantageously, providing two locks disposed at diametrically opposed locations may allow for opposing engagement forces of the first lock component with the second lock component when the clamp is in the closed configuration. Therefore, this arrangement may provide a stronger lock that is less likely to fail under strain.

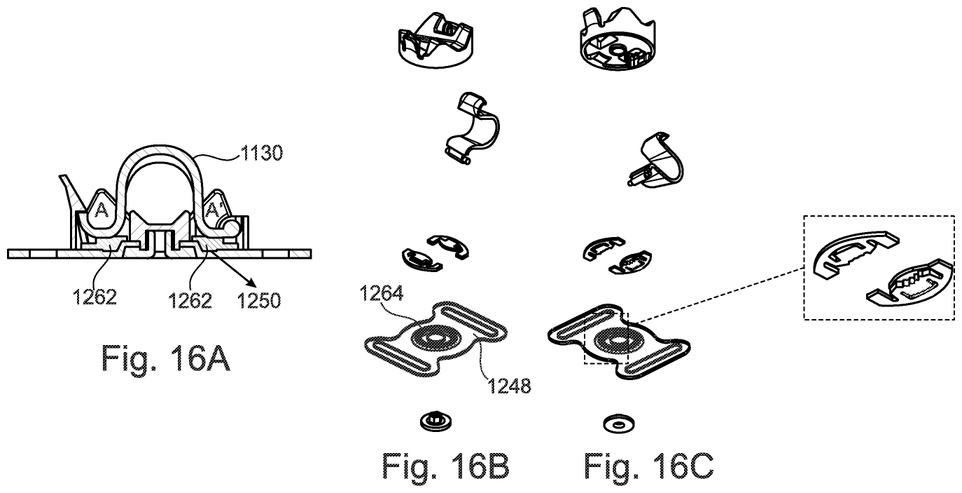

FIGS. 16A-16C show a portion of the medical article securement device 1200 according to another embodiment of the invention (FIG. 16A shows only the closed configuration). FIG. 16C shows an exploded view of the portion of the medical article securement device shown in FIG. 16A. In this embodiment, the securing arrangement is a clamp similar to that shown in FIGS. 15A-15D but with a slightly amended locking mechanism. Features of the securement device in common with the securement device 100 are provided with like reference numerals, incremented by 1100.

In this embodiment, the locking inserts 1262 of the first lock component are disposed within a tapered pocket 1250 of the insert 1248 of the body engaging portion. The locking inserts (shown enlarged in FIG. 16C) have a boss matching the shape of the tapered pocket 1250. This configuration provides a clearing distance between the first lock components 1262 and the second lock component 1264 in the open configuration. In other words, in the open configuration, the toothed features of the first lock component tabs 1262 and those of the second lock component (toothed ring) 1264 are not engaged. When moving to the closed configuration of the clamp 1230, the ends A, A′ of the clamp press against the locking inserts 1262, deflecting the cantilevered portion and sliding the boss along the tapered pocket 1250 until the teeth of the cantilevered tabs 1262 and the toothed ring 1248 engage. Advantageously, this configuration provides maximum engagement between the toothed interfaces/features of the first and second lock components in the closed configuration. This configuration also increases the height of the teeth, which allows for greater engagement and therefore for a stronger lock.

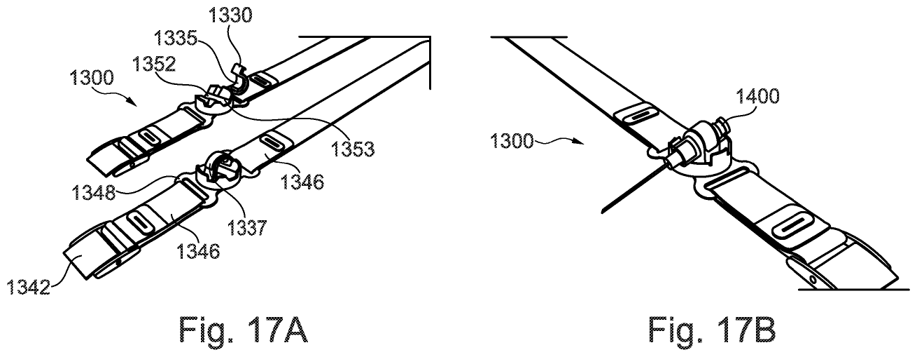

FIGS. 17A and 17B show renderings of a medical article securement device 1300 according to the invention. The securing arrangement in this embodiment is a clamp 1330. In FIG. 17B the device is shown holding a smart site of a cannula. Features of the securement device in common with the securement device 100 are provided with like reference numerals, incremented by 1200.

Device 1300 has a clamp 1330 as the securing arrangement. As clearly shown in FIG. 17A, the clamp 1330, which is fabricated from a hard material, comprises a resilient insert 1335 (e.g. made of rubber, silicone, medical grade silicone, resilient polymeric material, or the like). The resilient insert 1335 is configured to be in contact with the medical article 1400 when the clamp or snap cap is in the closed configuration (FIG. 17B). The resilient insert 1335 may allow the clamp or snap cap to secure medical articles which have slight variations in size (e.g. 0.1-2.5 mm variations in size). The medical article engaging portion 1352 also comprises a resilient insert 1353. The resilient inserts may have a high friction coefficient. The resilient inserts 1353, 1335 may be configured to absorb forces exerted on the medical article 1400 by movement of the patient to which the device and medical article are secured, and may resist movement of the medical article 1400 within the device 1300.

The clamp 1330 also comprises resilient material 1337 disposed on a top surface (which in use would be facing away from the medical article). The resilient insert 1335 of the clamp 1330 may be over moulded or extend through the material of the clamp 1330 to the top surface of the clamp. Advantageously, the resilient material disposed on a top side of the clamp 1330 may provide a textured touch point (e.g. soft/rubbery) on the upper side of the securing arrangement (clamp/snap cap). This may provide a clear tactile indication to the end user on where to push to close the securing arrangement and may assist patients with visual impairment who rely on tactile features. The resilient material disposed on the top surface of the clamp 1330 may comprise a luminescent material or a luminescent colouring. This may improve low-light visualisation of the medical article securement device (e.g. at night, or in low light conditions which are often encountered in the field).

In this embodiment, the body engaging member comprises two flexible straps 1346 which are coupled to hoops on the body engaging member insert 1348 and are provided with securing means (e.g. clasp) 1342 to secure the device around a body part of a patient.

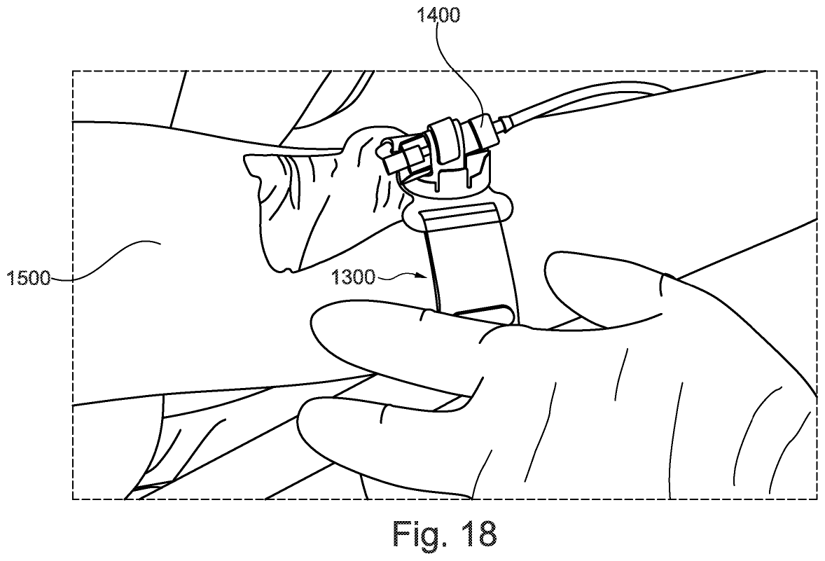

FIG. 18 is an image of a patient with a rendering of the device of FIGS. 17A and 17B as it would be used to secure an IV in a medical setting. In this case, medical article securement device 1300 is attached to an arm 1500 of a patient to secure a cannula 1400 (or smart site thereof) to the device 1300, to prevent rotational, longitudinal and/or lateral movement of the cannula 1400 secured within the device 1300.

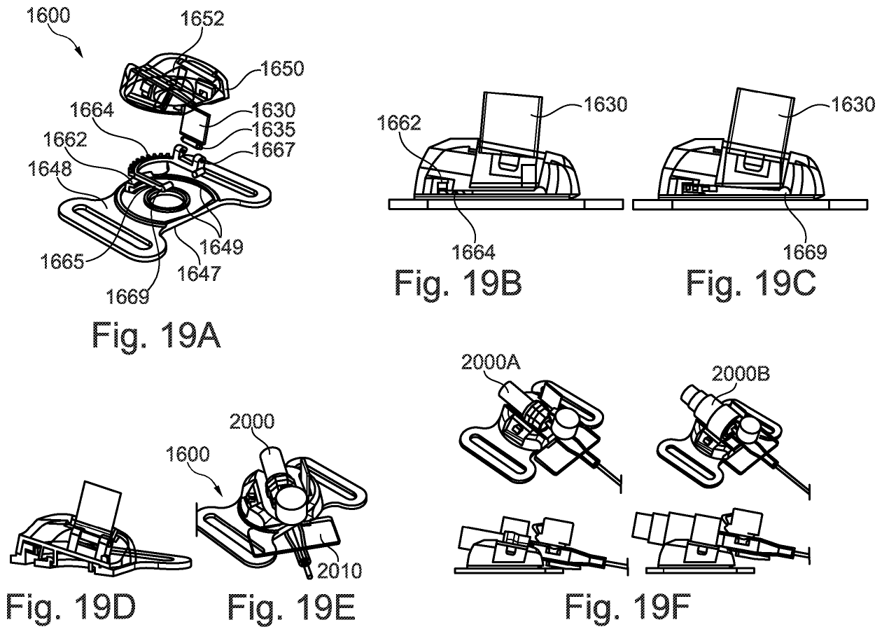

FIG. 19A shows an exploded view of a medical article securement device 1600 according to another embodiment of the invention. In this embodiment, the securing arrangement 1630 is a strap which is connected to a locking element 1665 disposed between the base of the medical article engaging member 1650 and the body engaging member 1648 (or insert or base thereof). The locking element 1665 carries the second lock component 1664 of the locking mechanism of the device 1600 (which in this embodiment is a toothed portion). The first lock component 1662 of the locking mechanism is formed on the base or insert of the body engaging member 1648. The first lock component 1662 is an inverted L with a toothed profile which is complementary to the toothed profile of the second lock component 1664 of the locking element 1665 (also L-shaped). When the device 1600 is assembled, the second lock component 1664 is configured to engage with the first lock component 1662 in the closed configuration of the device 1600. Advantageously, the L-shaped teeth structure on both lock components 1662, 1664 provides for a strong lock.

The securing arrangement (strap) 1630 is pivotally connected to the locking element 1665 at one end by means of a pin 1635 and yoke 1667 arrangement in this embodiment. When the device 1600 is assembled, the locking element 1665 is sandwiched between the base of the medical article engaging member 1650 and the base or insert of the body engaging member 1648. The base of the medical article engaging member 1650 is snap fitted on the base or insert of the body engaging member 1648 between raised annular lips 1649. This is advantageous as it provides a very strong connection of the parts, while allowing rotation of the base of the medical article engaging member 1650 relative to the body engaging member 1648.

The base of the medical article engaging member 1650 defines a channel or aperture through which the yoke portion 1667 of the locking element 1665 and/or the strap 1630 connected thereto pass (best seen in FIG. 19D). The strap 1630 (securing arrangement), the locking element 1665 and the base of the medical article engaging member 1650 are allowed to rotate as an assembled unit both clockwise and anticlockwise in this embodiment. In preferred embodiments, rotation of 30° in a clockwise and an anticlockwise direction is preferred as this provides a 60° range in which to adjust the position of the device to accommodate a medical article (e.g. NFC of a cannula) after it has been attached to a patient. The rotation may be free or it may occur in predetermined intervals/increments. In some embodiments, the rotation may occur in increments of about 10°, although the increment may be adjusted depending on the tuning requirements of the device.

FIGS. 19B and 19C shows side views of the device of FIG. 19A with a see-through detail of the locking mechanism of the device 1600 in the open configuration (when the securing arrangement 1630 is not tightened) and the closed configuration (when the securing arrangement 1630 is tightened) respectively. As seen in FIG. 19B, when the securing arrangement 1630 is not tightened (i.e. in the open configuration of the device 1600) the distance between the first lock component 1662 and the second lock component 1664 allows for clearance of the teeth of both lock components and the locking element 1665 and the base 1650 of the medical article engaging member can rotate about a rotational axis. In the closed configuration of the securing arrangement 1630 (i.e. when the securing arrangement 1630 is pulled towards the opposite side of the medical article engaging portion 1652 to tighten the securing arrangement 1630), the locking element 1665 is pulled upwards, thus reducing the distance between the first lock component 1662 and the second lock component 1664 such that the teeth engage and prevent further rotation of the locking element 1665 and the base 1650.

The locking element 1665 has two posts 1669 which engage the base of the medical article engaging member and act as pivot points about which the locking element 1665 is allowed to pivot when moving from the open configuration to the closed configuration of the device. As better seen in FIG. 19B, in the locking element 1665 rests flat against the base of the body engaging member 1648 in the open configuration, but in the closed configuration shown in FIG. 19C the locking element 1665 has pivoted about posts 1669, thus lifting the toothed profile 1664 of the locking element 1665 in order to engage with the toothed profile 1662 of the body engaging member 1648 to lock the device. The locking element may be allowed pivot (e.g. about 5°) between the open and closed configurations. Since the posts 1669 of the locking element 1665 are supported by the features of the base of the medical article engaging member 1650, the lock is robust under load. The locking element may engage with the base and/or the body engaging member in at least two opposing locations. This may lead to a stronger lock in the closed configuration.

The body engaging member may be secured to a body part of a patient by any suitable means. For example, in some embodiments, the body engaging member may be loaded onto an adhesive or otherwise secured with an adhesive to a body part of a patient. In other embodiments, the body engaging member 1648 may further comprise a strap (e.g. an overmoulded strap) covering the holes of the base of the body engaging member 1648. Using a strap is advantageous as it enables the device to be used multiple times on the same patient and be changed and/or cleaned between uses or between patients. An overmoulded strap is advantageous as it optimises flexibility, grip, and strength and stretch properties of the body engaging member.

FIG. 19D shows a cross-section of the device 1600 of FIGS. 19A, 19B, and 19C with a see-through detail of the annular snap arrangement between the base of the medical article engaging member 1650 and the body engaging member 1648, as well as the engagement between the base of the medical article engaging member and the locking element. The annular snap provides a very strong engagement between the base 1650 of the medical article engaging member and the body engaging member 1648, while allowing rotation. An annular snap arrangement may also reduce the number of parts required to assemble the device, and this in turn improves manufacturability and reduces the manufacture cost.

FIG. 19E shows a perspective view of the device of FIGS. 19A, 19B, 19C, and 19D hosting a needle free connector (NFC) for a cannula and shows how the device is configured to accommodate NFCs with wings and/or dressings adhered thereto. When the device 1600 is rotated, the cannula wing 2010 is forced to flex, or cannula or NFC 2000 can rotate about its longitudinal axis to enable wing 2010 and adhesive dressing to go underneath base of the body engaging member 1648. This is facilitated by the trimmed or cropped profile 1647 of one of the ends of the base body engaging member 1648 and one end of the medical article engaging member 1650.

FIG. 19F shows perspective and side views of the device of FIGS. 19A, 19B, 19C, 19D and 19E hosting NFCs of different sizes. For example, the NFCs 2000A on the left hand side images may be 10 mm diameter NFCs while those on the right hand side may be 15 mm diameter NFCs 2000B. Advantageously, a single medical article securement device can be used to accommodate medical articles (NFCs in this case) of different shapes and diameters. Although in these figures the medical article securement device is shown receiving NFCs of a cannula, the end user may secure the cannula/NFC at a different location. For example, once a cannula is inserted in a body part of a patient, the user may secure the cannula to the body part by securing the junction between the fluid line and NFC or between the fluid line and the Luer-lock/access point of the cannula with the device of the invention. This securement location may reduce the vertical tenting of the cannula and keep a leaner lower profile which would minimise or avoid disturbance of any adhesive dressing (e.g. Tegaderm®) applied to the cannula/wound site.

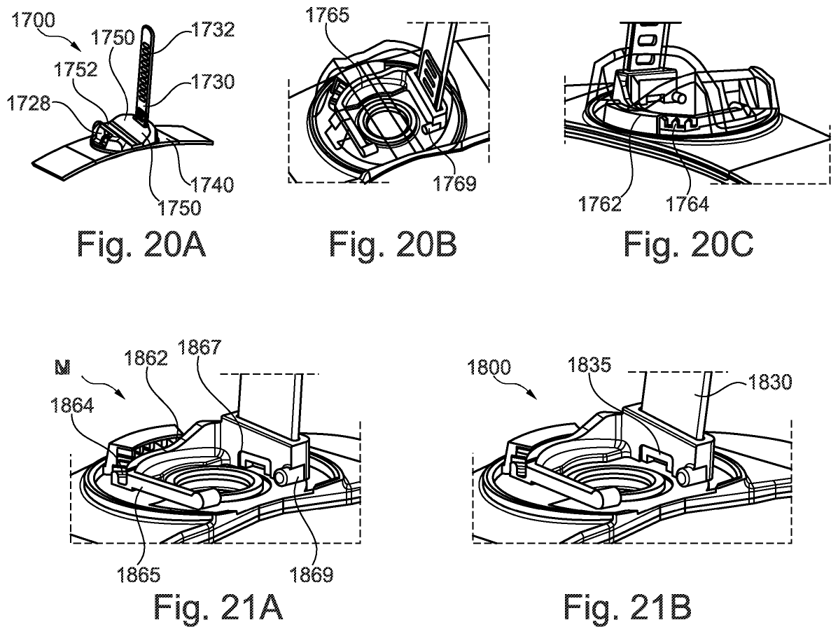

FIG. 20A shows a perspective view of another medical article securement device according to another embodiment of the invention similar to the embodiment of FIGS. 19A-F. This embodiment is very similar to that of FIGS. 19A-F, but it differs in the way the securing arrangement 1730 is connected to the locking element. In this embodiment, the securing arrangement is also a strap 1730 with apertures 1732 configured to be secured to a protrusion 1728 of the base 1750 of the medical article engaging member.

FIGS. 20B and 20C show top and side perspective views of the device of FIG. 20A with a see-through detail of the locking mechanism. As best seen in FIGS. 20B and 20C, the strap 1730 is fixedly connected to the locking element 1765 without a pivot (it can be clamped or otherwise fixedly secured to a rigid insert or rib), thus providing a more robust anchor than that of the device of FIGS. 19A-F.

FIGS. 21A and 21B show perspective views of the locking mechanism of a medical article securement device 1800 according to another embodiment of the invention in the open and closed configurations respectively. This device is very similar to those of FIGS. 19 and 20, but differs in that the strap 1830 is anchored to the locking element 1865 by means of an overmould/profile 1835 on the strap 1830 that is retained in a retaining profile 1867 of the locking element 1865. This engagement arrangement may provide for a more robust coupling as there is greater engagement surface between the locking element 1865 and the strap 1830.

Advantageously, the devices of FIGS. 19A-F, 20A-C and 21A-B provide a very robust lock for the rotation of the medical article engaging member since the lock is provided by engaging the first and second lock elements 1662/1664, 1762/1764, and 1862/1864 at the front of the device and the (pivot) posts 1669, 1769, 1869 and the corresponding features of the base of the medical article engaging member 1650, 1750 at the rear of the device. This double lock at the front and back ends of the device is able to withstand great rotational loads and forces. The securing arrangement for these devices may be fastened by any suitable means, such as zip ties, holes, or any other suitable fastening arrangement.

The embodiments of FIGS. 19A-F, 20A-C and 21A-B all employ a flexible and resilient securing arrangement (strap) that can adapt to different shape and sizes of cannulas or NFCs, therefore resulting in versatile devices that can accommodate different medical articles.

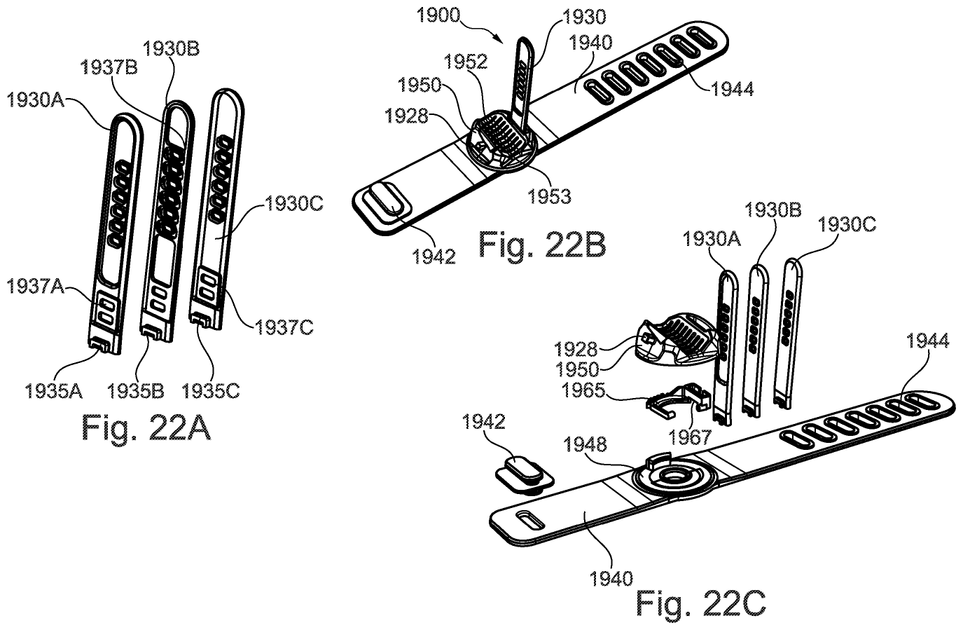

FIG. 22A shows a perspective view of three embodiments 1930A, 1930, and 1930C of the securing arrangement. Securing arrangement 1930C comprises a small rigid piece defining an overmould 1935C configured to engage a corresponding receiving feature on a base of the medical article receiving portion or a locking component of the device. The rigid piece may be manufactured from a rigid material such as plastic in order to provide a secure coupling of the securing arrangement (strap) 1930C and the base/locking element. The rigid piece also has a short rigid/semirigid rib 1937C configured to engage a resilient strap 1930C. The securing arrangement 1930A is similar but instead of a short rib, the securing arrangement 1930A comprises a semi-rigid rib 1937A running along the underside perimeter of the strap to provide a grip feature against the edges of the medical article (e.g. NFC) in use. The securing arrangement 1930B comprises a semi-rigid reinforcement structure in the form of a ladder structure 1937B extending along the length of the securing arrangement 1930 inside the strap to reduce stretch of the strap and reinforce it, thus increasing the capacity of the strap 1930B to withstand a loading force.

FIG. 22 B shows a perspective view of a medical article securement device according to an embodiment of the invention with a body engaging member 1940 in the form of a resilient strap (e.g. made of silicone) with apertures 1944 configured to allow the strap to be secured around body parts of different sizes when they engage male feature or protrusion 1942. The base 1950 of the medical article engaging member is formed of a hard material and it defines a medical article engaging portion 1952 for receiving a medical article (e.g. a cannula/Needle Free Connector/Luer Lock or the like). The medical article engaging portion 1952 is a tapered channel disposed at an angle. In this embodiment, it is lined with an insert made of a resilient material or insert 1953 defining ribs to provide better engagement with a medical article by increasing friction between the medical article and the insert in order to resist loads exerted on the medical article. In use, the medical article securing arrangement 1930, which is a resilient strap with apertures fixedly coupled to a locking element at one side of the medical article engaging portion 1952 and is configured to engage at the opposite side of the medical article engaging portion with a protrusion 1928.

FIG. 22C shows an exploded view of the medical article securement device 1900 of FIG. 22B with any one of the securing arrangement straps 1930A, 1930B, or 1930C of FIG. 22A. In this figure it can be best appreciated that, similar to the embodiments of FIGS. 19 and 20, the reinforcement section or insert 1948 of the body engaging member (strap 1940) has circular snap features for receiving the base 1950 of the medical article engaging member. There is a locking element 1965 sandwiched between the base 1950 and the insert/reinforcing element 1948 of the body engaging member. The locking element 1965 defines a retaining profile 1967 for receiving and securing the overmoulded feature 1935A, 1935B or 1935C of the securing arrangement 1930A, 1930B, or 1930C respectively.

Advantageously, the securing arrangements shown in FIG. 22A provide strong frictional contact between the channel and securing arrangement. The resilient material of the insert of the medical article engaging portion and the ribbed profile of said insert further increase the frictional contact between the securing arrangement, the medical article engaging portion, and the medical article. The securing arrangement must—in this configuration—have the elasticity to mould/grip around the medical article (e.g. NFC of different sizes) and have the resilience to not elastically deform to a loading force on the medical article (e.g. cannula), such as an upward and backward deflection.

For the avoidance of doubt, although the embodiments shown in the figures secure needle free connectors of cannulas in most figures, the device may be adapted to secure other medical articles. Furthermore, when the device is to be used to secure a cannula or any element of the cannula, an end user (e.g. medical practitioner) may decide which portion of the cannula needs to be secured with the device. For example, the device may contact and secure the cannula, the cannula's needle free connector (NFC) or the luer connector of the fluid line that engages the NFC or cannula. Advantageously, this enables the end user to decide which aspect of the IV connection needs to be secured. The universal nature of devices having resilient securing arrangements such as straps facilitates this versatility of the device.

In some instances, it may be beneficial to secure the junction between the luer connection and the NFC when the NFC is of a larger diameter, as this securement location mitigates any unwanted uplift of the cannula in terms of its vertical angle. For example, in the set-up of FIG. 19F (right), an end user would be encouraged to secure the line further back where the luer lock connects to the NFC to achieve the aforementioned effect.

All the devices of the invention provide a binary (open/closed or on/off) securing and locking mechanism that can be actuated with a simple movement in a single step or with a minimal number of steps. Once a medical article (e.g. cannula) is located in the desired place, the device of the invention can secure the medical article to a body part of the patient simply and efficiently without having to go through a lengthy process (e.g. screwing a bolt) to secure the device. This allows for non-skilled staff to secure the medical article to the patients, and minimises the risk of dislodgement of the medical article before it is secured with the device of the invention.

1. A medical article securement device, the device comprising:
a medical article engaging member comprising:
a base defining at least one medical article engaging portion configured to receive a medical article, and
a securing arrangement for reversibly securing the medical article to the base, the securing arrangement defining an open configuration and a closed configuration;
a body engaging member configured to affix the medical article securement device to a body part of a human or animal; and
a locking mechanism, wherein the locking mechanism is configured to allow rotation of the base of the medical article engaging member relative to the body engaging member about a rotational axis of the base when the securing arrangement of the medical article engaging member is in the open configuration, and
wherein the locking mechanism is configured to prevent rotation of the base of the medical article engaging member relative to the body engaging member about the rotational axis of the base when the securing arrangement of the medical article engaging member is in the closed configuration.

2. The medical article securement device according to claim 1, wherein the securing arrangement is a quick-release/quick-set securing arrangement, and/or wherein the securing arrangement is a non-screw fastening securing arrangement.

3. The medical article securement device according to claim 1, wherein the securing arrangement of the medical article engaging member defines a longitudinal axis and wherein the longitudinal axis of the securing arrangement is substantially perpendicular to the rotational axis of the base when the securing arrangement of the medical article engaging member is in the closed configuration.

4. The medical article securement device according to claim 1, wherein the securing arrangement comprises a clamp or at least one band.

5. The medical article securement device according to claim 1, wherein at least one of the securing arrangement and/or the base comprises a fastener configured to releasably secure the securing arrangement to the base.

6. The medical article securement device according to claim 1, wherein the securing arrangement is fixedly or pivotally coupled to the base or to a lock component of the locking mechanism at one side of the medical article engaging portion.

7. The medical article securement device according to claim 1, wherein the securing arrangement is a resilient strap having a first end and a second end, wherein the first end is fixedly or pivotally secured to the base or a lock component of the locking mechanism, and wherein the second end is configured to engage with an engagement site on the base for reversibly securing the securing arrangement to the base in the closed configuration and not to engage with the engagement site of the base in the open configuration.

8. The medical article securement device according to claim 1, wherein the securing arrangement is a clamp configured to fit around a medical article; the clamp having a first end and a second end, wherein the first end is pivotally coupled to the base and wherein the second end is configured not to engage with the base in the open configuration and to engage with the base in the closed configuration.

9. The medical article securement device according to claim 3, wherein in the open configuration the securing arrangement is configured to engage the base so as to allow movement of the securing arrangement relative to the base along the longitudinal axis of the securing arrangement, wherein movement of the securing arrangement relative to the base along the longitudinal axis of the securing arrangement is configured to modify a distance between the medical article engaging portion of the base and a section of the securing arrangement disposed opposite the medical article engaging portion of the base.

10. The medical article securement device according to claim 1, wherein:
the securing arrangement comprises at least one series of ratchet teeth and the base comprises at least one locking tooth configured to releasably secure the securing arrangement and the base and to allow movement of the securing arrangement relative to the base in at least the first longitudinal direction of the securing arrangement; or
the securing arrangement comprises at least one hole or a plurality of holes spaced apart along the longitudinal axis of the securing arrangement and the base comprises at least one hook, and wherein in the closed configuration, the hook is configured to engage the at least one hole of the securing arrangement to prevent movement of the securing arrangement relative to the base along the longitudinal axis of the securing arrangement.

11. (canceled)

12. The medical article securement device according to claim 1, wherein the body engaging member comprises a flexible material and a reinforced segment or insert resistant to stretching.

13. (canceled)

14. The medical article securement device according to claim 1, wherein the base defines at least one aperture, slit or channel configured to receive the securing arrangement and allow movement of the securing arrangement along a longitudinal axis of the securing arrangement through the at least one aperture, slit, or channel when the securing arrangement is in the open configuration.

15. (canceled)

16. The medical article securement device according to claim 1, wherein the medical article engaging portion comprises a channel, or a snap fit mechanism configured to engage a portion of the medical article and prevent longitudinal displacement of the medical article with respect to the base.

17. The medical article securement device according to claim 1, wherein the locking mechanism comprises a first lock component and a second lock component, and wherein the distance between the first lock component and the second lock component is greater when the securing arrangement is in the open configuration than when the securing arrangement is in the closed configuration.

18. The medical article securement device according to claim 17, wherein the locking mechanism comprises at least one of: a frictional lock between the lock components, a unidirectional or bi-directional ratchet mechanism, a series of teeth in the first lock component and a pawl on the lock component, a series of teeth in the first lock component and a corresponding series of teeth on the second lock component, at least one male feature in the first lock component and a corresponding female feature in the second lock component.

19. (canceled)

20. The medical article securement device according to claim 17, wherein the locking mechanism comprises a locking element disposed between the base of the medical article engaging member and the body engaging member, wherein the locking member is operatively coupled to the base of the medical article engaging member and to the securing arrangement.

21. The medical article securement device according to claim 20, wherein the locking member is configured to pivot between a first position when the securing arrangement is in the open configuration and a second position when the securing arrangement is in the closed configuration.

22. The medical article securement device according to claim 17, wherein the securing arrangement is a clamp and wherein one of:
first lock component is a toothed cantilever arm extending from the base substantially parallel to the body engaging member and disposed at a close distance from the second lock component, but allowing free rotation of the base with respect from the body engaging member when the clamp (or snap cap) is in the open configuration; and the second lock component is a toothed feature configured to engage with the toothed feature of the first lock component, and wherein the toothed feature of the second lock component is disposed on the body engaging member or on an insert or base to which the body engaging member is configured to be attached;
the first lock component is at least one cantilevered tab comprising a toothed feature disposed on a locking insert located between the clamp and the body engaging member or insert of the body engaging member, and wherein the second lock component is a toothed feature configured to engage with the toothed feature of the first lock component, and wherein the toothed feature of the second lock component is disposed on the body engaging member or on an insert or base to which the body engaging member is configured to be attached, optionally wherein the at least one cantilevered tab is disposed within a tapered pocket of the body engaging portion or insert thereof and wherein the at least one locking insert has a boss matching the shape of the tapered pocket;
the first lock component comprises at least one of a toothed feature and/or a male feature disposed on at least one of a first end and/or a second end of the clamp; and the second lock component comprises at least one of a toothed feature and/or a corresponding female feature disposed on the body engaging member or on an insert or base to which the body engaging member is configured to be attached.

23. (canceled)

24. (canceled)

25. (canceled)

26. (canceled)

27. (canceled)

28. The medical article securing device of claim 20, wherein the locking element comprises a first lock component configured to engage with a second lock component disposed on the base or on the body engaging member when the securing arrangement is in the closed configuration and not to engage with the second lock component when the securing arrangement is in the open configuration.

29. The medical article securing device of claim 21, wherein the locking member engages with the based and/or the body engaging member in at least two opposing locations.
